SEQUENCE LISTING (1 sequence)

SEQ ID NO: 1 - protein, 19 aa, Artificial sequence
  CDR1 of 16/6
GYYWSWIRQPPGKGEEWIG

(19) United States
(12) Patent Application Publication
(10) Pub. No.: US 2020/0338195 A1
(43) Pub. Date: Oct. 29, 2020

(54) PHARMACEUTICAL COMPOSITIONS COMPRISING 0.25 MG DOSE OF SYNTHETIC HUMAN PEPTIDES FOR TREATING SYSTEMIC LUPUS ERYTHEMATOSUS

(71) Applicant: XTL BIOPHARMACEUTICALS LTD., Raanana (IL)

(72) Inventors: Murray B. UROWITZ, Toronto (CA); David ISENBERG, London (GB); Daniel WALLACE, Studio City, CA (US); Monique BEN-AM, Ramat Gan (IL); Joshua LEVINE, Raanana (IL); Daphna PARAN, Even-Yehuda (IL)

(73) Assignee: XTL BIOPHARMACEUTICALS LTD., Raanana (IL)

(21) Appl. No.: 16/930,924

(22) Filed: Jul. 16, 2020

Related U.S. Application Data
(63) Continuation of application No. 15/673,555, filed on Aug. 10, 2017, now abandoned.
(60) Provisional application No. 62/373,376, filed on Aug. 11, 2016.

(51) Int. Cl. A61K 39/395; C07K 16/44; A61K 31/573; A61K 47/40; A61K 9/00; A61K 45/06; A61K 9/19

(52) U.S. Cl. CPC A61K 39/39583; C07K 16/44; A61K 31/573; A61K 2039/505; A61K 9/0019; A61K 45/06; A61K 9/19; A61K 47/40

(57) ABSTRACT

The present invention provides a method of treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B comprising periodic administration to the human subject of one subcutaneous injection of 0.25 mg dose of Edratide every week, so as to treat the human subject.

### CROSS-REFERENCE TO RELATED APPLICATIONS

This application is a continuation application of U.S. application Ser. No. 15/673,555, filed on Aug. 10, 2017, which claims priority to U.S. Application No. 62/373,376, filed on Aug. 11, 2016, which is incorporated herein by reference.

Throughout this application, various publications are referenced by full citations. The disclosures of these publications in their entireties are hereby incorporated by reference into this application in order to more fully describe the state of the art as known to those skilled therein as of the date of the invention described and claimed herein.

### SEQUENCE LISTING

The Sequence Listing submitted herewith is an ASCII text file (2020-07-16 Sequence Listing.text, created on Jul. 16, 2020, 654 bytes) via EFS-Web is hereby incorporated by reference.

### BACKGROUND OF THE INVENTION

Systemic lupus erythematosus (SLE), or lupus, is a debilitating autoimmune disease characterized by the presence of an array of autoantibodies, including antibodies to dsDNA, to nuclear antigens and to ribonucleoproteins (Petri M., Am Fam Physician, 1998). SLE predominantly affects women (at a 9:1 ratio), particularly during childbearing years, and has strong genetic and environmental components (Harley et al., Nat Genet., 2008).

SLE can affect almost any organ or system of the body (Fairhurst et al., Adv. Immunol., 2006) SLE may include periods in which few, if any, symptoms are evident (“remission”) and other times when the disease becomes more active (“flare”) (Gottschalk et al., Front Immunol., 2015).

SLE is characterized by autoreactive T and B lymphocytes that circulate in the blood and can be found in most body tissues. Contact between autoreactive T and B cells, which is mediated by proteins on the surface of both cell types, is required for pathogenic autoantibody production. The etiology of SLE remains unknown but is thought to be multifactorial, resulting from complex interactions between hormonal, genetic and environmental factors (Danchenko et al., Lupus. 2006).

The Systemic Lupus International Clinics (SLICC) classification criteria for SLE requires four criteria (with one having to be a clinical criterion and one having to be an immunologic criterion). The requirement for at least one clinical and one immunologic criterion reflects the opinion of SLICC that neither clinical criteria alone nor positive serologic tests alone should be considered SLE, as SLE is ultimately an autoantibody-driven clinical disease (Petri et al. Arthritis Rheumatology, 2012).

The revised American College of Rheumatology (ACR) requires 4 of 11 criteria to be met for classification of patients for a research definition of SLE diagnosis. These 11 criteria include malar rash, discoid rash, photosensitivity, oral ulcers, arthritis, serositis, pleuritis, or pericarditis, renal disorder, neurological disorder, haematological disorder, immunological disorder, and anti-nuclear antibody presence. The diagnosis is typically a clinical one predicated on accumulating information by performing a complete history, physical examination along with supporting laboratory findings (American College of Rheumatology, 1999).

Mild forms of SLE may be treated with antimalarial medications, non-steroidal anti-inflammatory drugs, and topical and/or low-dose glucocorticoids (Bailey et al., 2011). In addition, low-dose oral steroids or intramuscular injections of depot steroid preparations can be used for mild disease.

More severe cases of SLE may be treated with high-dose glucocorticoids and cytotoxic drugs to block cell proliferation and suppress the immune system (Bailey et al., 2011; Manson & Rahman, 2006; American College of Rheumatology, 1999). Life threatening, severely disabling manifestations of SLE are treated with high doses of oral glucocorticoids (1 mg/kg/day) and/or IV pulse methylprednisolone at doses of 500-1000 mg/day for 3 consecutive days. Undesirable effects of chronic glucocorticoids include an array of prominent adverse effects such as cushingoid habitus, central obesity, hypertension, infection, capillary fragility, hirsutism, accelerated osteoporosis, cataracts, diabetes mellitus, myopathy and psychosis (Ruiz-Irastorza et al. Rheumatology (Oxford). 2012).

Cytotoxic agents are used for controlling active disease, reducing the rate of disease flares, and reducing steroid requirements. Undesirable side effects include bone marrow depression, increased infections with opportunistic organisms, irreversible ovarian failure, alopecia and increased risk of malignancy (Ioannou et al., Postgrad Med J. 2002).

SLE is an inflammatory disease for which to date there is no definitive treatment or cure (Sthoeger et al., Journal of Autoimmunity 2014). The disease results in acute and chronic complications (Haubitz M., Biologics 2010). The currently available treatments may induce remission but do not cure the disease and have significant side effects. There is therefore an unmet need in this field, and both physicians and patients would welcome new treatments which could potentially eliminate or reduce the unwanted manifestations of the disease with fewer side effects.

Peptides based on the complementarity-determining region of the human monoclonal anti-DNA 16/6Id antibody capable of immunomodulating SLE associated responses have been disclosed in U.S. Pat. No. 7,858,738 and in PCT International Publication No. WO 02/067848 A2, the entire contents of which are hereby incorporated by reference. In particular, region CDR1 was found to inhibit the proliferative response of peripheral blood lymphocytes (PBL) of SLE patients to the human anti-DNA 16/6Id mAB, and to ameliorate disease manifestations of mice afflicted with spontaneous or experimental SLE. Human CDR1 is a synthetic peptide of 19 amino acids based on the complementarity-determining region 1 (CDR1) of the human anti-dsDNA mAb denoted 16/6 Id (Waisman, A., et al. Proc. Natl. Acad. Sci. U.S.A., 1997).

Edratide is the acetate salt of the synthetic peptide composed of 19 amino acid residues. Treatment of SLE patients using a parenteral subcutaneous (sc) dosage form was disclosed in U.S. Pat. No. 7,294,687, the entire contents of which are hereby incorporated by reference.

### SUMMARY OF THE INVENTION

The present invention provides effective doses of the peptide Edratide to treat populations of patients with SLE that decrease the amount of this peptide required for treatment without decreasing the beneficial activity.

The present invention provides a method of treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B comprising periodic administration to the human subject of one subcutaneous injection of 0.25 mg dose of Edratide every week, so as to treat the human subject.

The present invention also provides a method of treating a human subject suffering from systemic lupus erythematosus (SLE) and having SLEDAI-2K score of 6 or higher comprising periodic administration to the human subject of one subcutaneous injection of 0.25 mg dose of Edratide every week, so as to treat the human subject.

The present invention also provides a method of treating a human subject suffering from systemic lupus erythematosus (SLE) comprising periodic administration to the human subject of one subcutaneous injection of 0.25 mg dose of Edratide every week and an amount of corticosteroid drug in a manner effective to treat SLE.

The present invention also provides a method of reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, which comprises periodic administration to the human subject of one subcutaneous injection of 0.25 mg dose of Edratide every week and said reduced amount of corticosteroid drug in a manner effective to treat SLE.

The present invention also provides Edratide for use in treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B by periodic administration to the human subject of one subcutaneous injection of 0.25 mg dose of Edratide every week.

The present invention also provides Edratide for use in treating a human subject suffering from systemic lupus erythematosus (SLE) and having SLEDAI-2K score of 6 or higher by periodic administration to the human subject of one subcutaneous injection of 0.25 mg dose of Edratide every week.

The present invention also provides Edratide for use in treating a human subject suffering from systemic lupus erythematosus (SLE) by periodic administration to the human subject of one subcutaneous injection of 0.25 mg dose of Edratide every week and an amount of corticosteroid drug in a manner effective to treat SLE.

The present invention also provides Edratide for use in reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, by periodic administration to the human subject of one subcutaneous injection of 0.25 mg dose of Edratide every week and said reduced amount of corticosteroid drug in a manner effective to treat SLE.

The present invention also provides the use of Edratide in the manufacture of a medicament for treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B, wherein the medicament is prepared for periodic administration to the human subject of one subcutaneous injection of 0.25 mg dose of Edratide every week.

The present invention also provides the use of Edratide in the manufacture of a medicament for treating a human subject suffering from systemic lupus erythematosus (SLE) and having SLEDAI-2K score of 6 or higher, wherein the medicament is prepared for periodic administration to the human subject of one subcutaneous injection of 0.25 mg dose of Edratide every week.

The present invention also provides the use of Edratide in the manufacture of a medicament for treating a human subject suffering from systemic lupus erythematosus (SLE), wherein the medicament is prepared for periodic administration to the human subject of one subcutaneous injection of 0.25 mg dose of Edratide every week and an amount of corticosteroid drug in a manner effective to treat SLE.

The present invention also provides the use of Edratide in the manufacture of a medicament for reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, wherein the medicament is prepared for periodic administration to the human subject of one subcutaneous injection of 0.25 mg dose of Edratide every week and said reduced amount of corticosteroid drug in a manner effective to treat SLE.

In some embodiments, the human subject has at least one organ system categorized as BILAG A or at least two organ systems categorized as BILAG B.

In some embodiments, the human subject has SLEDAI-2K score of 6 or higher.

The present invention also provides a kit or package which comprises:
a) one or more unit doses of 0.25 mg dose of Edratide; and
b) instructions for use of a) for periodically dispensing 0.25 mg dose of Edratide to a human subject who is suffering from systemic lupus erythematosus (SLE), and
wherein the human subject has at least one organ system categorized by the British Isles Lupus Assessment Group 2004 (“BILAG”) as category A (“BILAG A”) or at least two organ systems categorized as BILAG B; wherein the human subject has SLEDAI-2K score of 6 or higher; wherein the human subject is administered an amount of corticosteroid drug; and/or wherein the amount of corticosteroid dose received by a human subject is reduced to an amount less than the amount received by the human subject for effective control of SLE disease activity when only the corticosteroid drug is administered to the human subject.

### BRIEF DESCRIPTION OF THE FIGURES

FIG. 1

Patient distribution over the course of the PRELUDE study.

FIG. 2A-2C

Disease activity as measured by BILAG responder analysis in different groups. FIG. 2A—BILAG responder analysis at Last Office Visit (LOV) vs Baseline predefined analysis on Intent to Treat (ITT) cohort (0.5 mg arm n=76; Placebo arm n=83). FIG. 2B—BILAG responder analysis of patients receiving steroids <20 mg/day at baseline (0.5 mg arm n=68; Placebo arm n=69) FIG. 2C—BILAG responder analysis of seropositive patients (anti-DNA>30 IU at baseline) (0.5 mg arm n=26 Placebo arm n=31).

FIG. 3

Medicinal flare analysis on Intention to Treat (ITT) cohort.

FIG. 4

SLE Composite Index (cSRI) on Intention to Treat (ITT) cohort.

FIG. 5

Edratide: Demographic Characteristics at Baseline by Treatment Group. At baseline, 84 patients in the 0.5 mg dose, 87 in the 1 mg dose, 82 in the 1.5 mg dose and 87 on placebo had either BILAG A or B. BILAG, British Isles Lupus Assessment Group; PRELUDE, A Study to Evaluate the Tolerability, Safety and Effectiveness of Edratide in the Treatment of Lupus; SLE, systemic lupus erythematosus.

FIG. 6

Steroids at baseline (given per Os, mg equivalent doses to prednisolone).

FIG. 7

Edratide common adverse events (AEs)—incidence presented by preferred/high-level term and dose and sorted by the risk ratio of pooled Edratide doses versus placebo.

FIG. 8

Endpoints with at least a trend for the 0.5 mg dose as compared with placebo (intention to treat (ITT) or <20 mg steroid dose cohort).

### DETAILED DESCRIPTION OF THE INVENTION

Previous clinical studies using Edratide to treat SLE did not test doses lower than a weekly injection of 0.5 mg per patient. The present invention discloses for the first time an effective dose of a weekly injection of 0.25 mg per patient.

The present invention further describes the successful reduction of steroid dose received by SLE patients, following treatment with a low weekly dose of Edratide. Reduction in the steroid consumption of a patient is beneficial as adverse effects (such as cushingoid habitus, central obesity, hypertension, infection, capillary fragility, hirsutism, accelerated osteoporosis, cataracts, diabetes mellitus, myopathy and psychosis) of chronic steroid treatment are common and undesirable.

The present invention provides a method of treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B comprising periodic administration to the human subject of one subcutaneous injection of 0.25 mg dose of Edratide every week, so as to treat the human subject.

The present invention also provides a method of treating a human subject suffering from systemic lupus erythematosus (SLE) and having SLEDAI-2K score of 6 or higher comprising periodic administration to the human subject of one subcutaneous injection of 0.25 mg dose of Edratide every week, so as to treat the human subject.

The present invention also provides a method of treating a human subject suffering from systemic lupus erythematosus (SLE) comprising periodic administration to the human subject of one subcutaneous injection of 0.25 mg dose of Edratide every week and an amount of corticosteroid drug in a manner effective to treat SLE.

The present invention also provides a method of reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, which comprises periodic administration to the human subject of one subcutaneous injection of 0.25 mg dose of Edratide every week and said reduced amount of corticosteroid drug in a manner effective to treat SLE.

The present invention also provides a method of treating a human subject suffering from systemic lupus erythematosus (SLE) comprising periodic administration to the human subject of one subcutaneous injection of 0.25 mg dose of Edratide every week, wherein at baseline of Edratide administration the human subject receives an amount of corticosteroid dose which is less than the amount required by the human subject for effective control of SLE disease activity when only the corticosteroid drug is administered to the human subject, so as to treat the human subject.

Some embodiments of the present invention further comprises gradually reducing the amount of corticosteroid hormone after baseline.

In some embodiments, the corticosteroid hormone is prednisone or prednisone equivalent.

In some embodiments, the amount of prednisone or prednisone equivalent is 15 mg/day or less.

In some embodiments, the amount of prednisone or prednisone equivalent is 1 mg/day or less, 2 mg/day or less, 3 mg/day or less, 4 mg/day or less, 5 mg/day or less, 6 mg/day or less, 7 mg/day or less, 7.5 mg/day or less, 8 mg/day or less, 9 mg/day or less, 10 mg/day or less, 11 mg/day or less, 12 mg/day or less, 13 mg/day or less, 14 mg/day or less or 15 mg/day or less.

In some embodiments, the amount of prednisone or prednisone equivalent at baseline is 15 mg/day or less.

In some embodiments, the amount of prednisone or prednisone equivalent at baseline is 1 mg/day or less, 2 mg/day or less, 3 mg/day or less, 4 mg/day or less, 5 mg/day or less, 6 mg/day or less, 7 mg/day or less, 7.5 mg/day or less, 8 mg/day or less, 9 mg/day or less, 10 mg/day or less, 11 mg/day or less, 12 mg/day or less, 13 mg/day or less, 14 mg/day or less or 15 mg/day or less.

Some embodiments of the present invention further comprises gradually reducing the amount of prednisone or prednisone equivalent to 7.5 mg/day or less.

Some embodiments of the present invention further comprises gradually reducing the amount of prednisone or prednisone equivalent to 1 mg/day or less, 2 mg/day or less, 3 mg/day or less, 4 mg/day or less, 5 mg/day or less, 6 mg/day or less, 7 mg/day or less, 7.5 mg/day or less, 8 mg/day or less, 9 mg/day or less, 10 mg/day or less, 11 mg/day or less, 12 mg/day or less, 13 mg/day or less, 14 mg/day or less or 15 mg/day or less.

In some embodiment, the reduced amount of prednisone or prednisone equivalent is 7.5 mg/day or less.

In some embodiment, the reduced amount of prednisone or prednisone equivalent is 1 mg/day or less, 2 mg/day or less, 3 mg/day or less, 4 mg/day or less, 5 mg/day or less, 6 mg/day or less, 7 mg/day or less, 7.5 mg/day or less, 8 mg/day or less, 9 mg/day or less, 10 mg/day or less, 11 mg/day or less, 12 mg/day or less, 13 mg/day or less, 14 mg/day or less or 15 mg/day or less.

In some embodiments, the human subject has been administered an amount of prednisone or prednisone equivalent for at least 14 days before baseline.

In some embodiments, the human subject has at least one organ system categorized as BILAG A or at least two organ systems categorized as BILAG B.

In some embodiments, the human subject has SLEDAI-2K score of 6 or higher.

In some embodiments, the treatment is effective to reduce lupus disease activity.

In some embodiments, the lupus disease activity is measured by a disease activity score selected from the group consisting of British Isles Lupus Assessment Group 2004 (BILAG), SLE disease activity index (SLEDAI-2K), SLEDAI-2K Responder Index 50 (SRI-50), composite SLE Responder Index (cSRI), minimum clinically important differences (MCID), patient reported short-form quality of life assessment (SF-36) Physical Component Summary (PCS) and/or Mental Component Summary (MCS), and lupus-specific quality of life form (Lupus-QOL) or a combination thereof.

In some embodiments, the treatment results in the subject who had at least one organ system categorized as BILAG A or at least two organ systems categorized as BILAG B at baseline, having reduction in the one organ system categorized as BILAG A or in the at least two organ systems categorized as BILAG B by one score in any one organ system, without having any other organ systems deteriorated to BILAG A or B.

In some embodiments, the treatment results in the subject who had at least one organ system categorized as BILAG A or at least two organ systems categorized as BILAG B at baseline, having all organ systems categorized as either BILAG C or BILAG D/E after treatment.

In some embodiments, the treatment results in a minimum clinically important difference (MCID) of one for SRI-50.

In some embodiments, the treatment results in the subject who had at least one organ system categorized as BILAG A or at least two organ systems categorized as BILAG B at baseline, having all organ systems categorized as either BILAG C or BILAG D/E after treatment, and no deterioration measured by SLEDAI-2K after treatment.

In some embodiments, the treatment results in the subject having equal or greater than 4 point improvement in the SELENA-SLEDAI, wherein the subject having no new organ system categorized as BILAG A or no more than one organ system categorized as BILAG B, and wherein the subject having less than 0.3 point increase in the physician global assessment.

In some embodiments, the treatment results in the subject having significant change in SF-36 PCS and/or MCS relative to baseline.

In some embodiments, the treatment results in the subject having increased time to first confirmed severe SLE flare or time to first confirmed major SLE flare.

In some embodiments, the treatment results in increased time to first confirmed severe SLE flare, and wherein a severe SLE flare comprises a subject having any new organ system categorized as BILAG A or having any two new organ systems categorized as BILAG B.

In some embodiments, the treatment results in increased time to first confirmed major SLE flare defined by the Fortin definition of major flare, which comprises initiation or increase of immunosuppressive or high-dose corticosteroids therapy, hospitalization or death due to SLE.

In some embodiments, the treatment results in the subject having a significant change in cumulative damage index as measured by Systemic Lupus International Collaborating Clinics/American College of Rheumatology Damage Index (SLICC/ACR DI).

In some embodiments, the treatment results in the subject having a significant change in daily glucocorticoid dose.

In some embodiments, the treatment results in the subject having a significant improvement in Lupus-QOL.

In some embodiments, the treatment results in the subject having significant improvement of global assessment of disease activity based on minimum clinically important differences (MCID).

In some embodiments, the subject has a documented medical history to meet Systemic Lupus International Clinics (SLICC) classification criteria for SLE, or has accumulated 4 classification criteria of the American College of Rheumatology for SLE.

In some embodiments, the subject has been on stable SLE treatment regimen for at least three months at baseline.

In some embodiments, the subject receives 2 g/day or less of mycophenolate mofetil.

In some embodiments, the subject receives 1440 mg/day or less of mycophenolate sodium.

In some embodiments, 0.25 mg dose of Edratide contains 0.25 mg of Edratide free base.

In some embodiments, 0.25 mg dose of Edratide contains about 0.275 mg of Edratide acetate.

In some embodiments, the subcutaneous injection of 0.25 mg dose of Edratide further comprises 120 mg of hepta-(sulfobutyl ether)-β-cyclodextrin.

The present invention also provides Edratide for use in treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B by periodic administration to the human subject of one subcutaneous injection of 0.25 mg dose of Edratide every week.

The present invention also provides Edratide for use in treating a human subject suffering from systemic lupus erythematosus (SLE) and having SLEDAI-2K score of 6 or higher by periodic administration to the human subject of one subcutaneous injection of 0.25 mg dose of Edratide every week.

The present invention also provides Edratide for use in treating a human subject suffering from systemic lupus erythematosus (SLE) by periodic administration to the human subject of one subcutaneous injection of 0.25 mg dose of Edratide every week and an amount of corticosteroid drug in a manner effective to treat SLE.

The present invention also provides Edratide for use in reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, by periodic administration to the human subject of one subcutaneous injection of 0.25 mg dose of Edratide every week and said reduced amount of corticosteroid drug in a manner effective to treat SLE.

The present invention also provides the use of Edratide in the manufacture of a medicament for treating a human subject suffering from systemic lupus erythematosus (SLE) and having at least one organ system categorized by the British Isles Lupus Assessment Group 2004 (“BILAG”) as category A (“BILAG A”) or at least two organ systems categorized as BILAG B, wherein the medicament is prepared for periodic administration to the human subject of one subcutaneous injection of 0.25 mg dose of Edratide every week.

The present invention also provides the use of Edratide in the manufacture of a medicament for treating a human subject suffering from systemic lupus erythematosus (SLE) and having SLEDAI-2K score of 6 or higher, wherein the medicament is prepared for periodic administration to the human subject of one subcutaneous injection of 0.25 mg dose of Edratide every week.

The present invention also provides the use of Edratide in the manufacture of a medicament for treating a human subject suffering from systemic lupus erythematosus (SLE), wherein the medicament is prepared for periodic administration to the human subject of one subcutaneous injection of 0.25 mg dose of Edratide every week and an amount of corticosteroid drug in a manner effective to treat SLE.

The present invention also provides the use of Edratide in the manufacture of a medicament for reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, wherein the medicament is prepared for periodic administration to the human subject of one subcutaneous injection of 0.25 mg dose of Edratide every week and said reduced amount of corticosteroid drug in a manner effective to treat SLE.

In some embodiments, the human subject has at least one organ system categorized as BILAG A or at least two organ systems categorized as BILAG B.

In some embodiments, the human subject has SLEDAI-2K score of 6 or higher.

The present invention also provides a kit or package which comprises:

a) one or more unit doses of 0.25 mg dose of Edratide; and

b) instructions for use of a) for periodically dispensing 0.25 mg dose of Edratide to a human subject who is suffering from systemic lupus erythematosus (SLE), and

wherein the human subject has at least one organ system categorized by the British Isles Lupus Assessment Group 2004 (“BILAG”) as category A (“BILAG A”) or at least two organ systems categorized as BILAG B; wherein the human subject has SLEDAI-2K score of 6 or higher; wherein the human subject is administered an amount of corticosteroid drug; and/or wherein the amount of corticosteroid dose received by a human subject is reduced to an amount less than the amount received by the human subject for effective control of SLE disease activity when only the corticosteroid drug is administered to the human subject.

All combinations of the various elements described herein are within the scope of the invention.

### Definitions

As used herein, “Edratide”, also denoted hCDR1, is the acetate salt of a synthetic peptide composed of 19 amino acid residues, in a sequence which is based on the complementarity determining region 1 (CDR 1) of a human anti-dsDNA mAb termed 16/6 Id.

The molecular formula of the peptide sequence is:

H-Gly-Tyr-Tyr-Trp-Ser-Trp-Ile-Arg-Gln-Pro-Pro-Gly-
Lys-Gly-Glu-Glu-Trp-Ile-Gly-OH

The structural formula of the peptide sequence is:

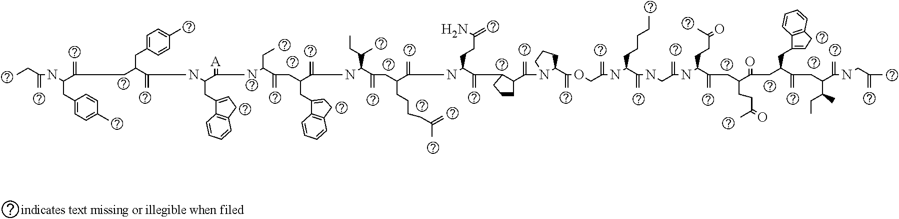

The molecular formula of Edratide free base is C₁₁₁H₁₄₉N₂₇O₂₈ (free base). The relative molecular mass is 2309.6 (as anhydrous, free base).

As used herein “hepta-(sulfobutyl ether)-β-cyclodextrin” includes sulfobutylether beta-cyclodextrin sodium (SBECD, CAPTISOL®). CAPTISOL® is a commercially available polyanionic β-cyclodextrin derivative with a sodium sulfonate salt separated from the hydrophobic cavity by a butyl ether spacer group, or sulfobutylether (SBE). CAPTISOL® is the trade name for Ligand Pharmaceuticals, Inc.'s hepta-substituted sulfobutylether β-cyclodextrin (SBECD) preparation (captisol.com). The structure of CAPTISOL® allows drug molecules to fit in the hydrophobic cavity, thereby isolating the drug molecule from the aqueous solvent. Because the outer surface of CAPTISOL® is hydrophilic, the solubility of the complexed drug molecule is thereby enhanced. The use of cyclodextrins to enhance the solubility of drug molecules is disclosed in U.S. Pat. Nos. 5,134,127 and 5,376,645, the entire contents of which are hereby incorporated by reference. Pharmaceutical composition comprising an aqueous carrier, 0.1 mg/ml to 2.5 mg/ml Edratide and 70 mg/ml to 170 mg/ml of hepta-(sulfobutyl ether)-β-cyclodextrin or a salt of hepta-(sulfobutyl ether)-β-cyclodextrin is disclosed in U.S. Pat. No. 7,294,687, the entire contents of which are hereby incorporated by reference.

As used herein, a subject at “baseline” is a subject prior to administration of Edratide.

As used herein, “BILAG” refers to the British Isles Lupus Assessment Group (BILAG) 2004, which is a disease index devised for patients with SLE based on the treating physician's intention to treat (Isenberg et al., 2005).

As used herein, the term “organ system” as used in connection with BILAG refers to the following 9 systems considered in BILAG 2004 index: constitutional, mucocutaneous, central nervous system, musculoskeletal, cardiovascular/respiratory, abdominal, renal and haematological. The BILAG 2004 assessment consists of 101 questions (and 5 additional items required mainly for calculation of glomerular filtration rate). Each question is answered as: 0=not present; 1=improving; 2=same; 3=worse and 4=new. The index records disease activity occurring over the past 4 weeks as compared with the previous 4 weeks. Based upon the scoring to each of these questions, a pre-defined algorithm, specific for each system, provides a disease activity score ranging from A to E for each system:

A=12, which is defined as severe disease requiring medium/large doses of corticosteroids (>20 mg prednisolone or equivalent) and/or starting or increasing immunosuppressive drugs, or high-dose anticoagulation (INR>3) (Yee et al., Rheumatology, 2010);

B=8, which is defined as disease activity requiring somewhat lower doses of immunosuppressives, e.g. <20 mg prednisolone, and/or specific drugs, such as anti-malarial, anti-epileptic, anti-depressant and NSAIDs, or topical steroids;

C=1, which is defined as mild persistent disease activity only requiring symptomatic treatment e.g. analgesics or NSAIDs;

D=0, which is defined as the organ or system was once active but is no longer so; and

E=0, which is defined as the organ or system was never active.

As used herein, SLE disease activity index “SLEDAI-2K” is a validated tool developed as a global assessment of disease activity in SLE patients (Gladman et al., 2002). It represents the consensus of a group of experts in the field of lupus research. The SLEDAI-2K assesses 24 descriptors (sixteen clinical manifestations and eight laboratory measures) in 9 organ systems. Descriptors are given different weights, based on clinical importance, with dichotomic score (present/not present within the previous 30 days). A descriptor must be attributed to active SLE or otherwise should not be scored. The SLEDAI-2K is intended to evaluate current lupus activity and not chronic damage. As used herein, “SLEDAI” and “SLEDAI-2K” are interchangeable.

As used herein, “deterioration” in the context of SLEDAI-2K means worsening of disease activity as measured by SLEDAI-2K.

As used herein, “SRI-50” is a SLE disease activity index comprising the same 24 descriptors, covering nine organ systems, which generates a total score and reflects disease activity over the previous 30 days as does SLEDAI-2K (Touma et. al., 2012). Each of the SRI-50 descriptors has a definition to identify 50% or more improvement and generates a score for the corresponding descriptor. Overall, SRI-50 is an index, developed to reflect partial important improvement in disease activity between visits.

As used herein, “minimum clinically important difference” (MCID) refers to patient derived scores that reflect changes in a clinical intervention that are meaningful for the patient.

As used herein, “composite SLE Responder Index” (cSRI) is a SLE disease index which incorporates two different systems: BILAG and SLEDAI-2K, defined as substantial response as measured by BILAG 2004 and no deterioration as measured by SLEDAI-2K which was developed specifically for the study “Prelude” before the more frequently used index of SRI-4 developed for the belimumab studies.

As used herein, “patient reported short-form quality of life assessment” (SF-36) is a widely validated generic patient questionnaire shown to be sensitive to change in a variety of chronic diseases: hypertension and cardiovascular disease, diabetes, pulmonary disease, low back pain, rheumatoid arthritis (RA) and osteoarthritis (Ware J E, et al. (1992) Medical Care 30:473-483). The SF-36 consists of 36 questions representing eight important health concepts, each of which is scored on an individual “domain” scale: Physical Functioning, Role-Physical, Bodily Pain, General Health, Vitality, Social Functioning, Role-Emotional and Mental Health (Ware et al. Medical Care, 1992). These eight scales can be aggregated into two summary measures: the Physical (PCS) and Mental (MCS) Component Summary scores.

As used herein, the “Systemic Lupus Erythematosus International Collaborating Clinics/American College of Rheumatology” (SLICC/ACR) is an index for accumulated organ damage (Dayal et al., Lupus 2002). SLE damage is defined as an irreversible change in organ or system that has been present for at least 6 months.

As used herein, “about” with regard to a stated number encompasses a range of +10 percent to −10 percent of the stated value. By way of example, about 100 mg/kg therefore includes the range 90-110 mg/kg and therefore also includes 90, 91, 92, 93, 94, 95, 96, 97, 98, 99, 100, 101, 102, 103, 104, 105, 106, 107, 018, 109 and 110 mg/kg. Accordingly, about 100 mg/kg includes, in an embodiment, 100 mg/kg.

By any range disclosed herein, it is meant that all hundredth, tenth and integer unit amounts within the range are specifically disclosed as part of the invention. Thus, for example, 1 mg to 50 mg means that 1.1, 1.2 . . . 1.9; and 2, 3 . . . 49 mg unit amounts are included as embodiments of this invention.

All combinations of the various elements described herein are within the scope of the invention.

### Previous Clinical Studies

Study 101 was a phase Ia, multicenter, randomized, double-blind, placebo controlled, single dose, four-arm study to assess the tolerability and safety of subcutaneous injection of Edratide in SLE patients. The study is disclosed in U.S. Pat. No. 7,294,687, the entire contents of which are incorporated by reference herein. The '687 patent does not disclose the use of a weekly dose of 0.25 mg Edratide for the particular sub-population of subjects with SLE relevant to the instant invention.

A single sc injection in the thigh of 0.5, 1 or 2.5 mg Edratide or placebo was given to 36 SLE patients (9 subjects received 0.5 mg Edratide, 9 subjects received 1.0 mg Edratide, 10 subjects received 2.5 mg Edratide and 8 subjects received placebo).

Edratide exhibited an overall lower (0.5 mg group: 33% of the subjects) or similar incidence of adverse events (AEs) in the 1 mg and 2.5 mg group (67% and 80%, respectively) compared to the placebo group (75%).

No noteworthy change of total SLEDAI-2K score was seen in any subject during the study, except for one subject in the 0.5 mg group, who had an increase from 2 to 10 points at Week 4 after the dosing (descriptors: hematuria and pyuria). This change corresponded to a mild to moderate flare according to the protocol definition. However, this change in total SLEDAI-2K score was not confirmed as a lupus flare by the investigator. The subject's SLEDAI score returned to baseline (SLEDAI-2K score: 2) at the termination visit without initiation of corrective therapy.

A second clinical trial, Study 102 was a phase Ib, multicenter, bi-national, randomized, double-blind, four-arm, placebo controlled, multiple dose study to assess the tolerability and safety of subcutaneous injection of Edratide in SLE patients and was conducted in France (26 patients) and UK (7 patients). The study is also disclosed in the '687 patent.

33 SLE patients with stable, mild to moderate SLE disease (fulfilling at least 4/11 classification criteria of the ACR and with a score between 1 and 10 inclusive on the SLEDAI-2K score) were recruited and received eight doses of the study drug. Edratide was injected sc twice weekly during a 4 week period, either 0.5, 1 or 2.5 mg Edratide or placebo (8 subjects received each 0.5 mg of Edratide, 8 subjects received 1 mg of Edratide, 9 subjects received 2.5 mg of Edratide and 8 subjects received placebo).

There was no prominent difference in the overall incidence of AEs between all treatment groups. The most common AEs reported in all treatment groups were injection site reaction, followed by joint related signs/symptoms and headaches. There were no specific AEs reported predominantly in the active treatment group compared to the placebo group. No clear pattern of dose response was seen for the injection site reactions, except for edema, pruritus and urticaria, which were reported only in the 2.5 mg group. All injection site reactions were classified as mild in severity and the majority resolved on the day of injection.

There was no prominent difference between the active treatment groups and the placebo group in the incidence of AEs during the 8-week follow-up period (off-drug). Approximately 50% of the subjects reported AEs in all treatment groups. Most affected system organ class was the musculoskeletal, including joint related signs and symptoms. There were no specific AEs reported predominantly in the active treatment group compared to the placebo group.

No SAE was reported throughout the study and no AEs leading to premature termination of the study. All reported AEs were classified as mild to moderate in nature, except for one severe AE, herpes zoster, which was reported by one female subject after the eighth dose administration.

The adjusted mean SLEDAI (AMS) score decreased (improved) in the range of −0.1 to −1.5 points in all treatment groups from baseline to termination visit. The majority of subjects had no change or an improvement in the total SLEDAI-2K score during the study. Two subjects in the 0.5 mg group and 2 subjects in the 2.5 mg group had an increase in total SLEDAI-2K score of 0-3 points during the study. The increased scores were due to descriptors already found at screening but not present at the baseline visit and included increased DNA binding or arthritis or low complement or mucosal ulcers. Two subjects in total, 1 in the 2.5 mg group and 1 subject in the placebo group had an increase in total SLEDAI score of 3 to 12 points (experienced a new flare).

No trend in the mean group total BILAG score was apparent in any treatment group during the study. The majority of subjects had E (no activity ever), D (inactive disease) or C (mild stable disease activity) scores for all organ systems and stayed stable during the study. New onset of a B score (intermediate activity) in the musculoskeletal organ system was seen in two subjects, one in the placebo group and one for whom a flare was confirmed according to the protocol. In addition, a few subjects had a new onset of a B score in one organ system with no difference between the treatment groups. Affected organ systems included general system, musculoskeletal, mucocutaneous, hematological and neurological system.

Two subjects (6%), one in the 2.5 mg group and one in the placebo group, each experienced one new flare during the study, classified as mild to moderate in severity according to the protocol definition.

### EXPERIMENTAL DETAILS

### Example 1

### Prelude Study

The PRELUDE Study was a phase 2, multinational, multicenter, randomized, double-blind, placebo-controlled, multiple-dose, parallel group study to assess the efficacy, tolerability and safety of three doses of subcutaneously injected Edratide in SLE patients. The study is disclosed in Urowitz et al. 2015, the entire content of which is incorporated by reference herein.

The total study treatment duration was 26 weeks. Scheduled visits were performed at screening, baseline and at weeks 4, 8, 12, 16, 20, 24, and 26 (termination). During the first 8 weeks of the study, the protocol requested that baseline steroid dose be kept stable (no more than 30% reduction), while during the subsequent period the protocol suggested a gradual tapering-down scheme to a minimum of 7.5 mg prednisolone per day. Non-compliance with the tapering down scheme was not regarded as a protocol violation. During the entire course of the study it was allowed to increase the steroid prednisolone dose to up to 60 mg. Use of intravascular/intramuscular steroid was considered a protocol violation. The use of immunosuppressive drugs during the study was not allowed and resulted in early termination. Edratide and CAPTISOL® pharmacokinetic (PK) profiles were characterized in a small group of patients. PK assessment of CAPTISOL® for all four treatment groups was conducted following the first dose and Weeks 12 and 26 doses. Disease and drug related immunology parameters were tested at baseline and at several time points thereafter.

Subjects found eligible to participate in the study were randomized in a 1:1:1:1 ratio into one of the following four treatment groups:
0.5 mg Edratide (including 120 mg/vial CAPTISOL®)
1 mg Edratide (including 120 mg/vial CAPTISOL®)
2.5 mg Edratide (including 120 mg/vial CAPTISOL®)
Placebo for Edratide (including 120 mg/vial CAPTISOL®)

The main inclusion criteria of this trial were:
SLEDAI 2K score of 6-12
Stable concomitant medications (prednisone<40 mg, NSAIDs, antimalarials) for at least 1 month prior to baseline.

The main exclusion criteria for this trial were:
Active CNS Lupus
Active Lupus nephritis (protein to creatinine ratio above 0.75 and/or active urine sediments)
Treatment with azathioprine, methotrexate within 4 weeks prior to baseline or treatment with any other immunosuppressive drugs within 3 months prior to baseline or change in dose of anti-malarial drugs, in the last month prior to study start, any investigational drug, immunomodulating or cytotoxic drug in the 3 months prior to study start.

The co-primary end-points presented in their hierarchical order below were:
Change from baseline to last observed value (LOV) in SLEDAI 2K score (Landmark Analysis.)
Adjusted Mean SLEDAI 2K (AMS). AMS represents the time weighted average of disease activity as measured by SLEDAI 2K score [area under the curve (AUC) of SLEDAI 2K].

Secondary end-points:
BILAG proportion of responders. BILAG responders were defined as:
   a. Non Responder (NR)—same BILAG score in Last Observed Value (LOV) as in baseline in all systems or new A or B in at least one system compared to baseline (other systems may improve or deteriorate).
   b. Substantial Responder (SR)—all systems at LOV are either C or D/E providing that at least one system was either A or B at baseline
   c. Partial Responder (PR)—all complementary situations to NR and SR (at least one system improved, and at least one system did not improve from A or B at baseline to C or D/E at LOV, and no other system deteriorated from C or D/E at baseline to A or B at LOV).
SF-36 [Quality of Life (QoL)] Mental component Score. Change from baseline to LOV
SF-36 (QoL) Physical component Score—Change from Baseline to LOV
Time Weighted Average (TWA) on cumulative steroids dose with p.o. administration.

Exploratory end-points:
Primary end-point sensitivity analysis to steroid increase (excluding the observations following steroid increase)
Proportions of Zero SLEDAI Score at LOV
Time to Zero SLEDAI Score
BILAG Total Score—Change from Baseline to LOV

Flares Analysis:
Time to medicinal flare
Time to new BILAG A or at least two new B's
Time to new BILAG A or at least two new B's (excluding persistent activity)
Steroid dose (PO)—Change from baseline to LOV
PGA—Physician's Global Assessment

### Results

Overall exposure to study drug was comparable among the three groups. A total of 37, 39, 38 and 42 patient-years on Edratide 0.5 mg, 1.0 mg, 2.5 mg and placebo, respectively were exhibited with a median ranging from 181 to 183 days in all groups. Overall, a total of approximately 8636 mg of Edratide were administered during the study.

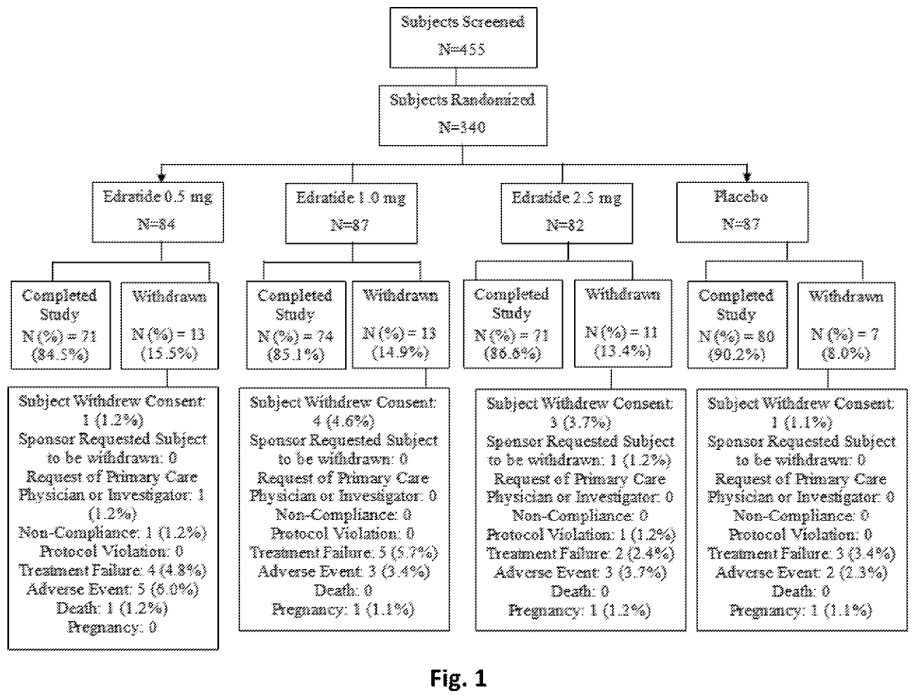

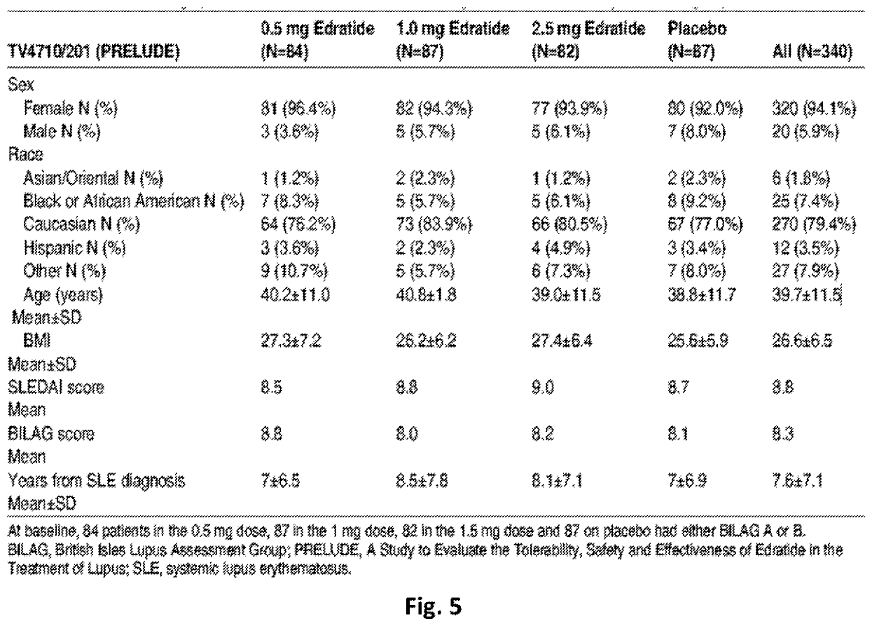

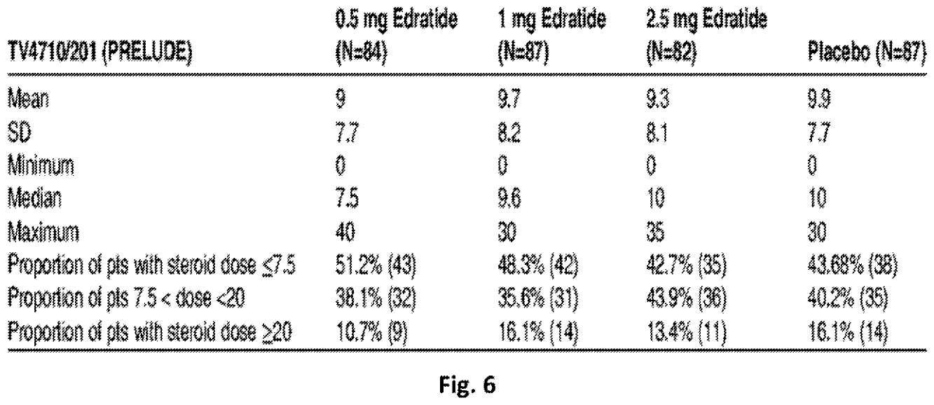

Four hundred-fifty five (455) subjects screened for this study, 115 were screening failures. The reasons for non-enrollment included: 70.4% did not meet inclusion/exclusion criteria, 20% decided to withdraw, and for 9.6% the reason was recorded as ‘Other’. Three hundred and forty subjects (340) were randomized into the study, where 84 were randomized into the Edratide 0.5 mg group, 87 into the Edratide 1.0 mg group, 82 into the Edratide 2.5 mg group, and 87 subjects to placebo. Thirteen subjects on Edratide 0.5 mg, 13 subjects on Edratide 1.0 mg, 11 subjects on Edratide 2.5 mg and seven subjects on placebo prematurely terminated the study. FIG. 1 shows the subgroups of subjects in the study. Demographic characteristics are summarized in FIG. 5. There was no statistical difference between the treatment groups in terms of steroids at baseline (FIG. 6).

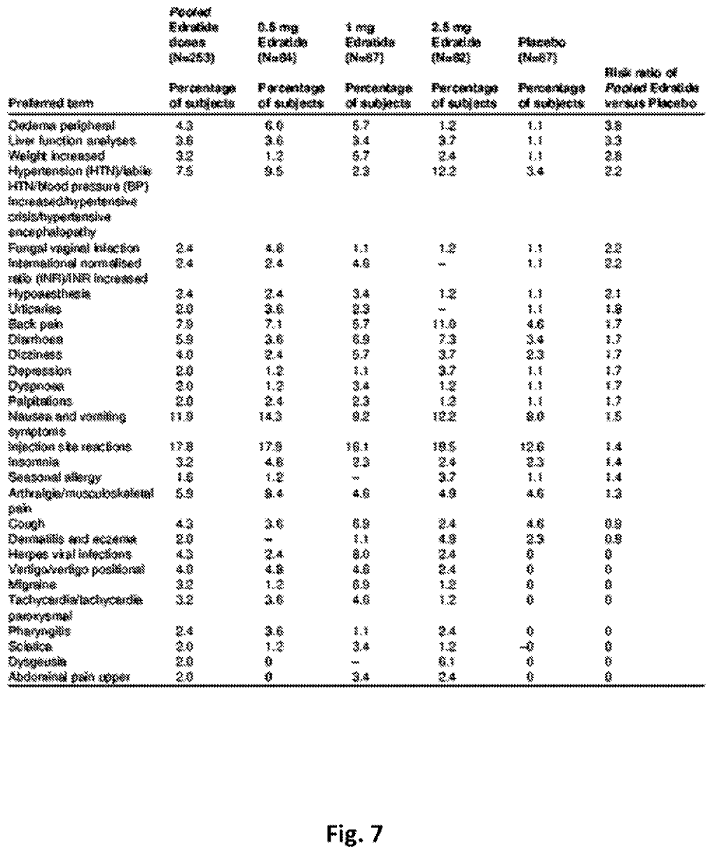

AE incidence was similar in all groups: 82.1%, 77%, 75.6% and 79.3% on Edratide 0.5 mg, 1 mg and 2.5 mg and placebo, respectively (FIG. 7).

Early withdrawal due to AE was reported for 5 subjects (6.0%) in the Edratide 0.5 mg group, 3 subjects (3.4%) on Edratide 1.0 mg, 3 subjects (3.7%) on Edratide 2.5 mg and 2 subjects (2.3%) in the placebo group. Two deaths occurred during the study. One death occurred during the study in the Edratide 0.5 mg group and another occurred 3 months after early termination (withdrew consent) in the placebo group.

Demographic characteristics: The four groups were comparable in the distribution of age, sex, race and BMI.

### Efficacy:

Primary endpoints: The study failed to meet its co-primary endpoints. For both endpoints, there were no differences in any of the active treatment groups compared to placebo. Overall, a mean reduction from baseline SLEDAI-2K score of about 35% was noted regardless of treatment.

Secondary endpoints: In further analysis, Edratide 0.5 mg seemed to have advantage over placebo on the first secondary end-point of the study, the BILAG responder analysis. 36% of the subjects treated with Edratide 0.5 mg had a substantial response, while only 22% of the placebo subjects were substantial responders (p=0.03, odds ratio (OR)=2.09). This result is not statistically significant following the hierarchical approach and correction for the overall type I error (multiplicity adjustment for the 3 tested doses of Edratide).

Post hoc analysis on BILAG responders suggests that patients on 0.5 mg Edratide showed improvement mainly in the cutaneous system. Edratide 1 mg showed a trend toward increasing the proportion of substantial responders as compared to placebo [p=0.07, without a correction for multiple comparison OR=1.85), but to a lesser extent than the 0.5 mg dose.

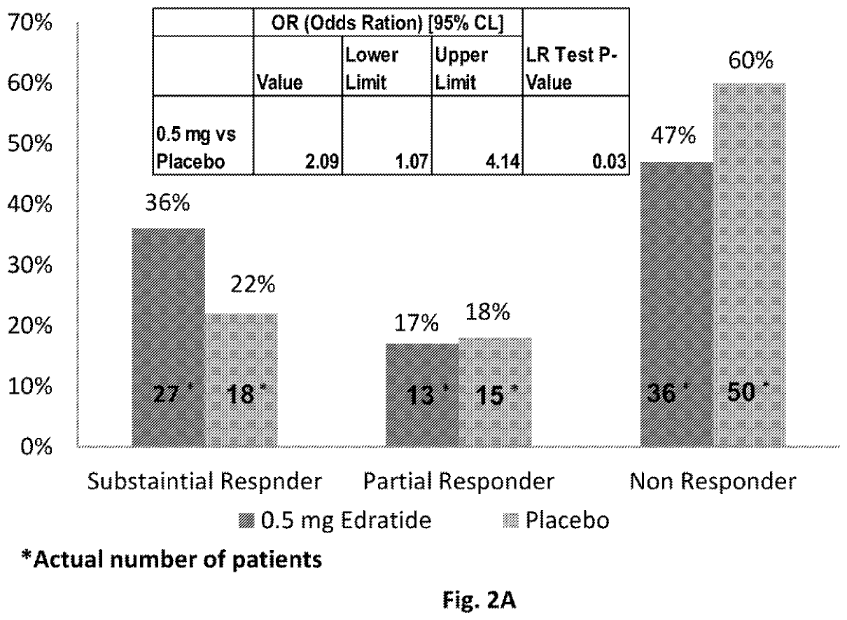

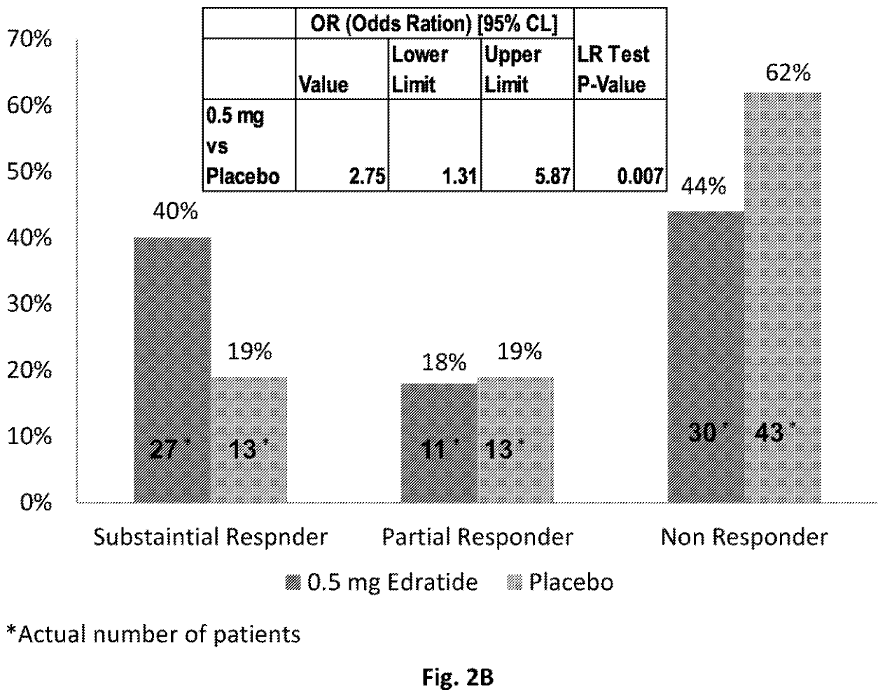

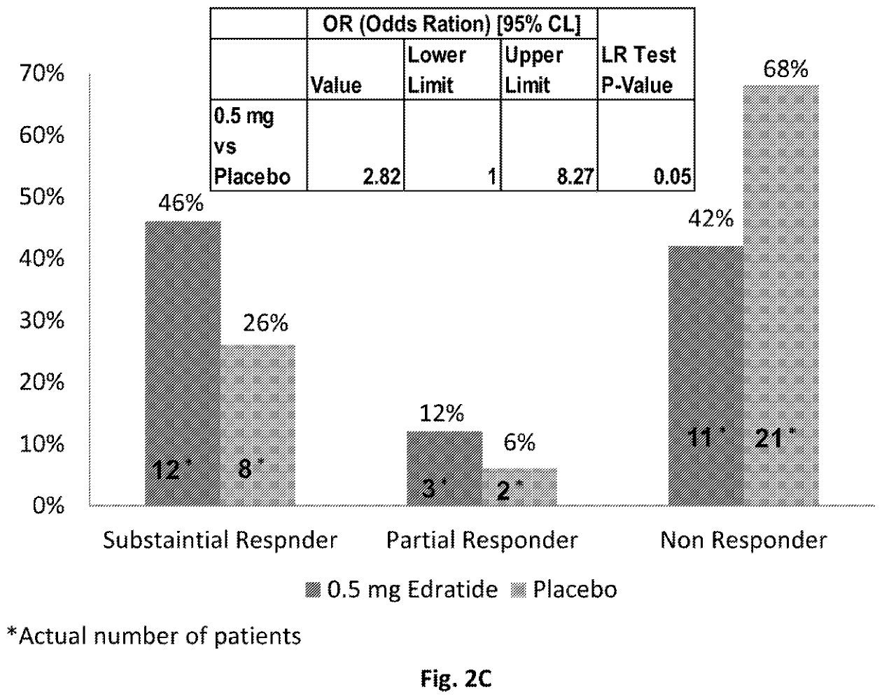

When the predefined definition of BILAG substantial responders was applied to the pooled cohort of 292 subjects (85% of the ITT cohort), with prednisolone dose <20 mg at baseline, 40% of the subjects treated with Edratide had a substantial response while only 19% of the placebo arm were substantial responders (p=0.007, OR=2.75) (FIG. 2B).

In a small group of patients who received no steroids at baseline and who were treated with 0.5 mg Edratide, 54% of the Edratide treated subjects were substantial responders as opposed to 13% of the placebo group (p=0.05).

A clinically significant effect was identified in seropositive patients (anti-DNA>30 IU). 46% of the substantial responder group were treated with Edratide as compared to 26% with placebo. In addition, 68% (n=21) of the non-responder group were treated with placebo, while 42% (n=11) (p=0.05) were treated with Edratide (FIG. 2C).

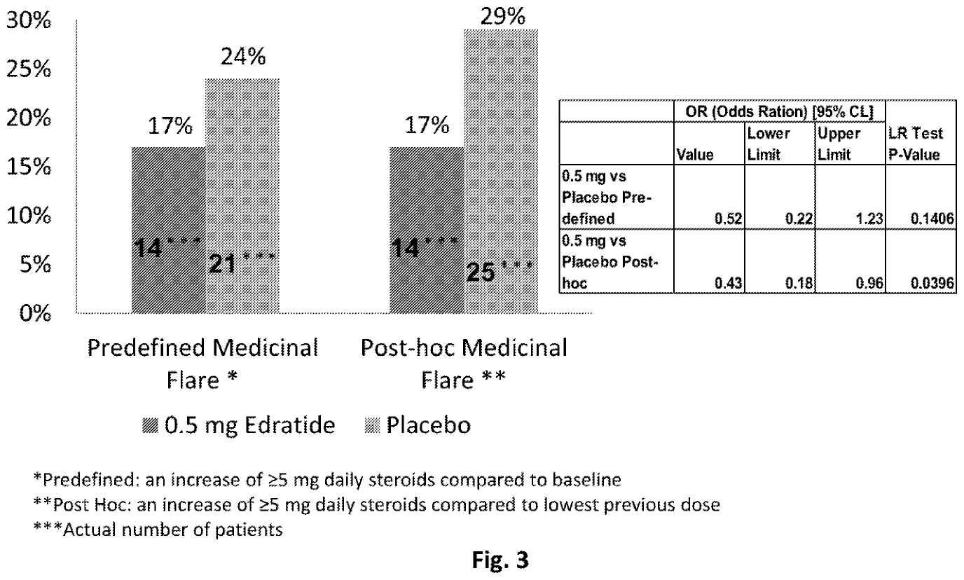

In the ITT group, post hoc analysis of Flare scoring showed that while 17% (n=14) of subjects on Edratide 0.5 mg had a medicinal flare, 29% (n=25) of the subjects on placebo flared (p=0.039, OR=0.43) (FIG. 3).

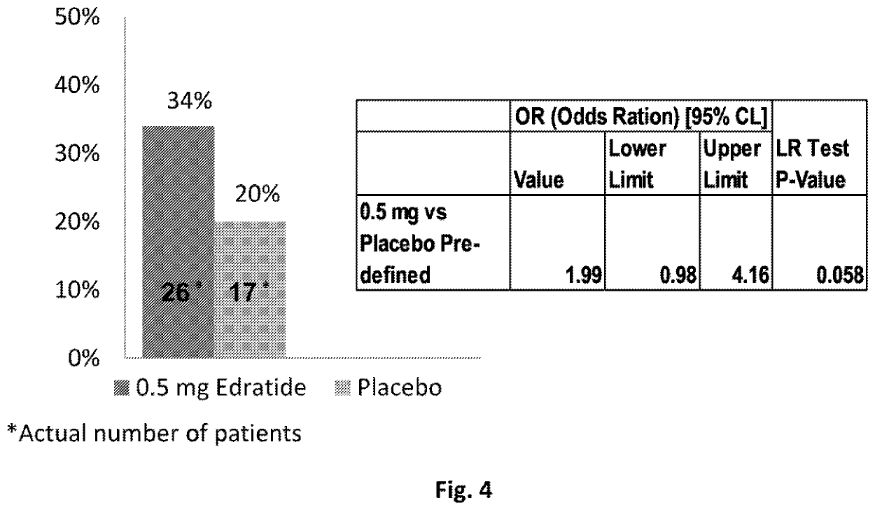

In addition, cSRI analysis substantiated the positive effects of Edratide with 34% (n=26) of the Edratide group being positive responders as compared to 20% (n=17) of the placebo group (p=0.058) (FIG. 4).

Covariate analysis did not find relation between response and body weight.

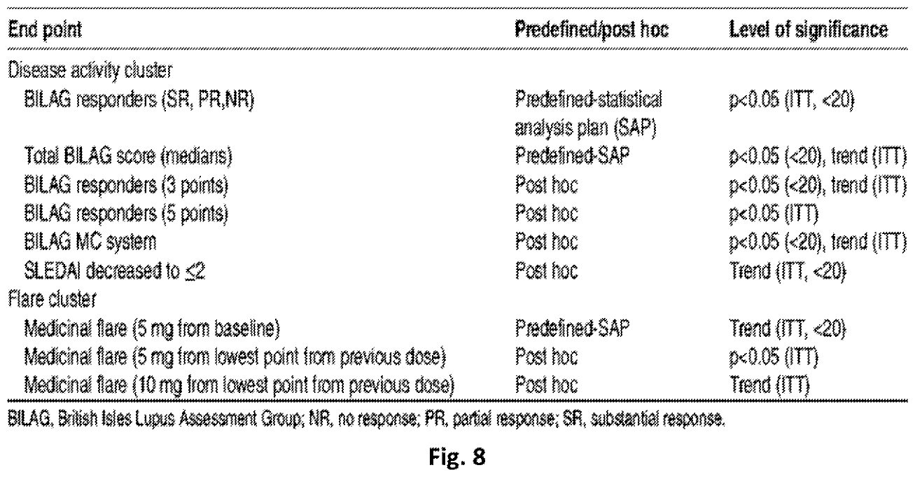

The disease activity clusters that showed at least a trend towards response are summarized in FIG. 8.

No clinically or statistically significant differences were noted between the treatment groups on the SF-36® mental or physical scores. While the change in the mental score was slightly better for Edratide 0.5 mg compared to placebo, the opposite trend was noted for the physical score.

No clinically or statistically significant differences were noted between the treatment groups for the cumulative change in steroid dose throughout the study. The mean baseline steroids dose for all treatment arms was 9-10 mg/day of prednisolone. It should also be noted that approximately 20% of subjects were steroid-free at baseline.

Safety: The incidence of adverse events (AEs) was similar in all groups: 82.1%, 77%, 75.6% and 79.3% on Edratide 0.5 mg, 1 mg and 2.5 mg and placebo, respectively. There was no clear dose response and no significant safety signal detected from the AEs analysis and the additional safety data. The majority of AEs reported with a risk ratio of at least 1.5 (in at least one Edratide group), compared to placebo, were in low incidence (<5%) and could be attributed to the primary disease or pre-existing medical conditions. The most common AEs reported for Edratide-treated subjects were herpes viral infections, vertigo and liver function analyses. Of the 11 subjects on Edratide with herpes viral infections, 8 had herpes simplex and 3 had herpes zoster [2 with a medical history of herpes zoster and one with ongoing NIDDM]. Most cases were seen in the 1 mg group: 5 with oral herpes and 2 with zoster. One subject on 2.5 mg discontinued the study prematurely due to mild AE of herpes zoster, reported on day 79.

Two deaths occurred during the study: A 42-year old woman from the 0.5 mg Edratide group died during the study, most probably due to septic shock. Another subject, a 60-year old woman from the placebo group died from the consequences of severe sepsis secondary to intestinal perforations and fecal peritonitis after she had withdrawn her consent and stopped study drug. Both deaths were classified by the investigators as not related to the study drug.

Serious Adverse Events (SAEs) were reported by 6 (7.1%) subjects from the 0.5 mg Edratide group, 10 (11.5%) subjects from the 1.0 mg dose group, 8 (9.8%) from the 2.5 mg dose group and 9 (10.3%) subjects from the placebo group. SAE incidence for the pooled Edratide group was similar to placebo, 9.5 vs. 10.3%. Most common for both, Edratide and placebo were infection-related SAEs attributable to SLE.

Early terminations due to AEs occurred more often on Edratide than on placebo.

### Example 2

### Low Dose Edratide Therapy

### Study Objectives

The objectives of this trial are:
To assess the efficacy, tolerability and safety of a once weekly administration of Edratide in patients with active SLE in comparison to placebo; all of whom are receiving active standard of care (SOC) therapy.
To identify a safe and effective dose of Edratide.
To study the pharmacokinetics of Edratide.

### Investigational Plan

### Overall Study Design and Plan—Description

A multi-national, randomized, double-blind, placebo-controlled, dose-ranging 26-Week Phase 2 study to assess the safety, efficacy and tolerability of Edratide administered subcutaneously to subjects with active SLE.

The study will test about 200 study subjects, in about 50 centres. The study population will include seropositive SLE patients (presence of anti-dsDNA antibodies by FARRzyme immunoassay, titer ≥30 IU, and/or the Crithidia luciliae immunofluorescence test, and/or antinuclear antibodies (ANA) ≥1:160 or 1:80 in which case the Adjudication Committee would review the subject's data at baseline.

The total duration of the study is expected to be 34 weeks:
Screening Phase: <30 days (4 weeks)
Phase 2 double-blind treatment phase: 26 weeks (6 months)
Follow-up phase for safety: 4 weeks

Study subjects will be randomly assigned (1:1:1) into three treatment groups to receive placebo, 0.25 mg or 0.5 mg of Edratide subcutaneously (s.c.) once weekly.

Study Phases:
Screening Phase: Each subject will be screened <30 days prior to Baseline visit and will undergo screening evaluations.
Randomization: Eligible subjects will be equally randomized to one of the 3 treatment groups (1:1:1) as follows:
1) Group A: Placebo
2) Group B: Edratide for s.c. injection 0.25 mg
3) Group C: Edratide for s.c. injection 0.5 mg
Treatment Phase: All subjects will receive a once weekly s.c. injection of placebo, 0.25 mg or 0.5 mg Edratide.
Safety follow-up Phase: All subjects will be followed up for safety for 30 days following last study treatment.

Glucocorticoid dosing: It is highly recommended to initiate tapering from the baseline dose of prednisone/prednisone equivalent on or after week 4, as clinically indicated from doses ≥15 mg/day to ≥7.5 mg/day; tapering could continue until week 18. No changes in dose of prednisone/prednisone equivalent doses are allowed during weeks 19-26, the last 8 weeks of the trial. No specific tapering regimen is required, but to be determined as clinically indicated by the investigator.

Evaluations during study: Subjects will attend the study sites for a screening visit, baseline visit and evaluation visits at Months 1, 2, 3, 4, 5, and 6 after starting treatment followed by a safety assessment/end of study visit. The total number of visits per patient will be 9.

A Data Safety Monitoring Board (DSMB) will be established.

Recruitment might be held during the Interim Analysis.

Active SLE patients will be screened for up to 30 days prior to the baseline visit (visit 2). Each subject will undergo screening evaluations. At baseline, eligible subjects will be equally randomized (1:1:1) to one of the following treatment groups:
Group A: Placebo for Edratide (120 mg CAPTISOL®) reconstituted in 1 ml Water for injection (WFI), or
Group B: Edratide for injection 0.25 mg (formulated in 120 mg CAPTISOL®) reconstituted in 1 ml WFI, or
Group C: Edratide for injection 0.5 mg (formulated in 120 mg CAPTISOL®) reconstituted in 1 ml WFI.

At the baseline visit, subjects will be administered the study drug and trained to self-administer the study drug at subsequent weekly administrations at home. After the administration of the first dose of study drug, subjects will remain under observation at the study site for at least 2 hours to ensure their well-being. Subjects will return to the study site for evaluations at the end of each month as outlined above.

### Termination of the Study

Termination of the study will be at the end of 34 weeks (screening, active treatment and safety follow-up), or if a safety signal appears and associated causality suggests that continuation of the study is futile, or if the interim analysis determines it is futile. Furthermore, the study can be discontinued at the discretion of the sponsor.

### Selection of Study Population

The study will include about 200 subjects with active SLE each of whom must have a documented medical history to meet Systemic Lupus International Clinics (SLICC) classification criteria for SLE or meet 4 or more of the of the revised (1997) American College of Rheumatology (ACR) criteria for the classification of SLE (Hochberg M C, 1997) by the time of the Screening visit. The SLE patients must meet all of the inclusion and none of the exclusion criteria (sections 6.3.1 and 6.3.2, respectively). At the Screening visit, all subjects must give their written informed consent to participate in the study prior to any study procedures being performed.

### Inclusion Criteria

The subjects must meet the following inclusion criteria:
1.Subjects diagnosed with SLE must have a documented medical history to meet Systemic Lupus International Clinics (SLICC) classification criteria for SLE, or have accumulated 4 classification criteria (1997 revised) of the American College of Rheumatology (ACR) for SLE prior to first dose.
2. Subjects with active SLE must have:
   a. At least one BILAG-A and/or two BILAG-B's, and
   b. SLEDAI score of 6 or higher, excluding serology scores, and be
   c. Serologically-positive as defined by 2 positive ANA or anti-dsDNA antibody test results (titer ≥30 IU by FARR radioimmunoassay and/or the Crithidia luciliae immunofluorescence test, and/or ANA 1:160 or 1:80 in which case the adjudication committee would review the subject's data) of which ≥1 test result must be obtained during screening
3. Subjects must be ≥18 years of age
4. SLE patients on stable SLE treatment regimen for at least three (3) months
5. SLE patients on 15 mg of prednisone/prednisone equivalent, which have been stable for at least 14 days
6. Subjects receiving 2 g/days of Mycophenolate mofetil (MMF) or ≤1440 mg of Myfortic
7. Women of child-bearing potential must practice a medically acceptable method of contraception. Acceptable methods include: surgical sterilization, IUD, hormonal preparations, or double barrier method (e.g. condom or diaphragm with spermicidal)
8. Subjects must be willing and able to give written informed consent prior to entering the study.
9. Subjects must understand the requirements of the study and agree to comply with the study protocol.

### Exclusion Criteria

The presence of any of the following excludes a subject from study enrollment:
i. Subjects with active Lupus nephritis as defined by a protein to creatinine ratio limit of ≥0.75 and/or active urine sediments defined as hematuria (>10 RBC/HPF) and/or RBC casts.
ii. Subjects with active central nervous system (CNS) Lupus as defined by CNS BILAG-A or BILAG-B.
iii. Subjects with active peripheral nervous system (PNS) Lupus as defined by PNS BILAG-A or BILAG-B.
iv. Subjects receiving >15 mg of glucocorticoid at baseline
v. Subjects having recently received
   a. Cyclophosphamide (wash-out period 3 months),
   b. Cyclosporine (wash-out period 3 months),
   c. Rituximab (wash-out period at least 52 weeks provided that the CD19 count has returned to normal), or
   d. Belimumab (wash-out period 6 months)
   e. Any investigational drug (wash-out period 5 half-lives of elimination)

Following the respective wash-out periods these subjects can enter the study.
vi. Subjects receiving >2 g/day of Mycophenolate mofetil (MMF) or >1440 mg of Myfortic
vii. Dialysis within the last month or scheduled to receive dialysis
viii. Previous kidney transplant or planned transplant
ix. Subjects with creatinine clearance of ≤30 ml/min
x. Subjects with hemoglobin <8.5 gm/dl or neutrophils <1000/mm3, or platelets <50,000/mm3
xi. Presence or history of:
   Congenital or acquired immunodeficiency
   Lymphoproliferative disease or previous total lymphoid irradiation
   A positive laboratory test for Hepatitis B surface antigen (HbsAg), HBcore Ab, HCV antibody active CMV infection within 3 months prior to randomisation or HCV antibody, or known HIV, infection
   Current infection requiring IV antibiotics or patients with an active infection requiring hospitalization in the last 30 days
   Severe cardiovascular disease including congestive heart failure
   Coexisting disease requiring systemic glucocorticoid, bone marrow insufficiency unrelated to active SLE (according to investigator judgment)
   Any bone marrow insufficiency
   Any overlapping autoimmune condition for which the condition or treatment of the condition may affect the study assessments or outcomes (e.g., scleroderma with significant pulmonary hypertension; any condition for which additional immunosuppressive therapy is indicated).
xii. Subjects with a history of malignancy, except subjects with non-invasive cervical neoplasia, basal cell or squamous cell carcinoma of the skin more than 5 years prior to Screening visit.
xiii. Diagnosis of drug-induced lupus ever.
xiv. Other major physical or psychiatric illness, or major traumatic injury within 6 months prior to randomization.
xv. Subjects who received any investigational medication within 3 months prior to randomization.
xvi. Known hypersensitivity or contraindication to sulfobutyl ether beta-cyclodextrin sodium (SBECD, CAPTISOL®) or other cyclodextrins, Edratide, glucocorticoid or any components of these drugs.
xvii. Women who are pregnant, breast feeding or intend to become pregnant within 6 months of last dose of study drug.
xviii. Inability or unwillingness to comply with the requirements of the protocol as determined by the investigator.
   Removal of Subjects from Therapy or Assessment

Subjects may withdraw from the study treatment at any time and be counted as treatment failure.

A subject will be withdrawn from study treatment if:
A deterioration in renal disease occurred based on a 30% or greater increase in 2 successive measurements of serum creatinine separated by at least 4 weeks (1 month)
He/she requires prohibited therapy
He/she requires more than the maximum allowed doses/courses of adjunctive SLE care
Patient and/or subject decision
Failure to repeatedly comply with study requirements

### Rescue Therapy While on Study

Rescue from ineffective therapy refers to prompt removal of subjects whose clinical status worsens. Rescue therapy should ensure that the subjects would not remain untreated with the study drug while their disease is poorly controlled. Rescue therapy can be employed in any case deemed necessary by the investigator. Rescue therapy can consist of any drug required for the subject's welfare. Early escape will be used in the case the physician considers the clinical condition unmanageable with the current protocol requirements.

A marked deterioration in any system such as rapid development of a major renal crisis or serious CNS involvement or any other medical condition, which at the discretion of the investigator requires institution of rescue therapy.

Subjects who require such rescue therapy will be deemed treatment failures.

### Replacement Policy

Withdrawn patients will not be replaced and will be part of the ITT analysis population.

### Study Product

### Description of Study Product

The study drug is a sterile, lyophilized powder contained in an amber glass single-use 2 ml vial with a rubber stopper. Edratide for injection doses correspond to 0.25 and 0.5 mg of Edratide free base in 120 mg of CAPTISOL®.

Edratide for injection and the corresponding placebo will be manufactured and packaged in compliance with the current GMP principles and guidelines applicable to investigational medicinal products.

Sterilized water for injection(s), which serves as the solvent/diluent for the preparation of the final injectable reconstituted solution, is provided in a separate vial.

### Description of Comparator Product

Placebo for Edratide for injection vials contain 120 mg of CAPTISOL®. All vials appear identical to keep the double-blinding nature for the study.

### Packaging and Labeling

Vials of Edratide for injection or the matching placebo will be packed as vials in a subject's visit pack. At each scheduled visit (except for the Screening and Termination visits), the subject will receive one monthly pack. The amount of study drug dispensed will suffice until the following scheduled visit. Packs will be assigned to subjects by using IVRS.

The label of the visit pack will include a fixed information section to include product name, storage conditions, dosing instructions etc. and a variable information section. The variable information section will include the following variable data: batch code, pack number and expiration date (all pre-printed) and blank fields for the investigator's name and subject number. These blank fields will be filled in manually by the investigator or pharmacist upon dispensing the pack to the subject.

### Conditions for Storage and Use

Study drug must be kept in a secure, limited-access, refrigerated (2°-8° C.) and controlled storage area. Only authorized personnel will have access to the study drug supplies.

The study site personnel at each site will be responsible for correct storage and handling of the study products. The drug supplies will be stored safely and properly in a temperature-controlled refrigerator (2°-8° C.), and must be kept under the prescribed conditions while the sterilized water for injection(s) in the separate vial will be stored at room temperature. The subjects should be instructed to store the study drug (in their original package) ideally in a refrigerator, but it can also be kept at ambient temperature.

The study drug pack will be dispensed to the subject at the study site at each scheduled visit starting at the baseline visit (except for the termination visit).

The subjects will be instructed to administer the study drug once weekly at the same day at approximately the same time of the day. The packs will be assigned to subjects by using an Interactive Voice Response System. After assigning a pack number to the subject, the investigator/pharmacist/coordinator should fill in manually the subject's number and investigator's name and visit number on the label of the dispensed monthly pack, as well as on the detachable parts, which will be attached to the subject source file.

Subjects will be instructed to return all the empty vials and unused study drug vials in their original packs.

### Method of Assigning Subjects to Treatment Groups

At the Screening visit, the subject will be assigned a screening number through the Interactive Voice Response System (IVRS). After meeting the inclusion/exclusion criteria, assignment of a subject to a treatment group will be performed by the IVRS according to the randomization scheme produced by a statistical expert. At the Baseline visit, each subject will be allocated a number in sequential chronological order per site. This number will replace the screening number. The IVRS will assign a pack number (per the subject's treatment arm), the subject will be dispensed with study drug labeled with the IVRS pack number. The IVRS will perform allocation of medication packs at monthly visits 1, 2, 3, 4, and 5.

### Dispensing, Compliance and Accountability

The subject will be requested to return all study drugs which includes all empty and unused vials (excluding ancillary supplies) in the original packs to the study site at every visit.

Compliance with the dosing regimen will be determined by performing accountability of returned study drug. The number of returned vials will be counted and recorded in the eCRF by site personnel.

The subjects will be trained on self-injection of the study drug (SC injection) in alternate sites (thigh, buttocks, abdomen or arm).

The subject number, pack number, batch code number, quantity of study drug returned by the subject and visit date will be recorded by the site personnel.

The study drug accountability records must be maintained at the site at all times.

Compliance will be calculated as a percentage by calculating the number of used vials divided by the number of total vials expected to be injected, multiplied by 100. Subjects with less than 75% overall compliance during the entire study will be considered non-compliant, although they will continue to be followed-up and included in the ITT cohort. Subjects who fail to comply with the study requirements may be withdrawn from the study, following consultation with the sponsor.

The investigator is responsible for maintaining accountability for the receipt, dispensing, and return of all study medication.

### Prior and Concomitant Therapy

Concomitant medications will be reviewed and recorded at every visit. Concomitant medications (other than glucocorticoids) are expected to be taken at a constant dose throughout the study.

### Disallowed Prior Medications

The following treatments may be used at doses stable for at least 3 months prior to baseline (first study drug administration) as medically indicated, according to the judgement of the investigator. No new starts of these drugs nor change of dose are allowed after randomization:
Oral azathioprine
Methotrexate
Mycophenolate mofetil (MMF) at <2 g/day or ≤4 tablets (1440 mg) of Myfortic
Leflunomide at doses ≤20 mg/day
Hydroxychloroquine or other anti-malarial drugs

Any subject initiated on any of the above mentioned immunosuppressive/antiproliferative agents after receiving the first dose of study drug, or having their dose increased during the study will be considered a treatment failure.

The baseline glucocorticoid dose will not exceed 15 mg/day prednisone/prednisone equivalent.

Subjects requiring “stress doses” of glucocorticoids are allowed to receive these for up to 7 days once in the study with the exception of the last 8 weeks of study treatment (weeks 19-26 of the treatment phase of the study). Subjects with exacerbations in disease activity (deterioration from baseline not meeting treatment failure criteria) are allowed to receive up to double their baseline prednisone/prednisone equivalent doses, but not more than an increase of 10 mg of prednisone/prednisone equivalent over their baseline dose (to a maximal dose of 25 mg) for up to 7 days once in the study at any time prior to the last 8 weeks on study treatment. Subjects entering the study without any prednisone/prednisone equivalent are allowed up to 10 mg of prednisone/prednisone equivalent for up to 7 days once in the study up until week 18 inclusive. One intra-articular glucocorticoid injection is allowed, provided that the prednisone/prednisone equivalent dose is not increased. No changes in dose of prednisone/prednisone equivalent are allowed during weeks 19-26, the last 8 weeks of the treatment phase of the trial.

Any increases in prednisone/prednisone equivalent doses for more than 7 days at any time during the study, or any increase in prednisone/prednisone equivalent after week 18 or any dose increase above a maximal dose of 25 mg of prednisone/prednisone equivalent will be considered a treatment failure and the patient will be considered a non-responder. They may continue in the protocol for safety evaluations.

### Disallowed Medication During the Study

The following drugs may not be taken during the study:
Cyclophosphamide
Cyclosporine
Rituximab
Belimumab

Any subject being treated with any of the above mentioned therapies during the study will be considered a treatment failure.

### Definition of Treatment Failure

Treatment failure is defined as any new BILAG A or any new BILAG B score requiring an increase in corticosteroid dose for more than 7 days and/or need for any rescue treatment during the study.

### Symptomatic Treatment

Symptomatic agents such as analgesics, NSAIDs, and topical preparations (including topical immunosuppressive preparations) are allowed at clinically appropriate doses.

### Efficacy and Safety Assessments

### Primary Efficacy Variable(s)

The primary efficacy variable is the proportion of subjects achieving a response at 26 weeks by BILAG 2004 in at least one organ system that was an A or B at baseline without deterioration to an A or B in any other system defined as;
One BILAG A or BILAG B scores dropping by one score in one system without deterioration in BILAG scores in any other system.

The British Isles Lupus Assessment Group (BILAG) (Isenberg et al., 2005) is a disease index devised for patients with SLE which is based on the treating physician's intention to treat. It was originally developed and subsequently validated by making agreed assumptions about the likely treatment that will be given to patients with particular clinical features in eight body systems. In its revised version, BILAG 2004, 9 systems have been devised. These include constitutional, mucocutaneous, central nervous system, musculoskeletal, cardiovascular/respiratory, abdominal, renal and haematological.

### BILAG 2004 Scores:

| Score | Definition |
|---|---|
| A | Severe disease requiring medium/large doses of |
| | corticosteroids (>20 mg prednisolone or equivalent) and/or |
| | starting or increasing immunosuppressive drugs, or high- |
| | dose anticoagulation (INR >3) |
| B | Disease activity requiring somewhat lower doses of |
| | immunosuppressives, e.g. <20 mg prednisolone, and/or |
| | specific drugs, such as anti-malarial, anti-epileptic, |
| | anti-depressant and NSAIDs, or topical steroids |
| C | Mild persistent disease activity only requiring symptomatic |
| | treatment e.g. analgesics or NSAIDs |
| D | The organ or system was once active but is no longer so |
| E | The organ or system was never active |

A BILAG assessment consists of 101 questions (and 5 additional items required mainly for calculation of glomerular filtration rate). Each question is answered as: 0=not present; 1=improving; 2=same; 3=worse and 4=new. The index records disease activity occurring over the past 4 weeks as compared with the previous 4 weeks.

A numerical coding scheme has been devised and recently, a modification to the Classical BILAG scoring system, more appropriate for the BILAG 2004 index, has been proposed (Yee et al., Rheumatology, 2010):
A=12
B=8
C=1
D/E=0

FDA in its guidance for industry on developing medical products for the treatment of SLE (June 2010) has declared BILAG the preferred index to study reduction in disease activity in clinical trials.

BILAG 2004 will be assessed at the baseline visit and every monthly visit thereafter.

### Secondary Efficacy Variables

Secondary efficacy variables in the order of analysis consist of the following:
Proportions of subjects achieving a substantial response by BILAG 2004 (BILAG 2004 SR) at week 26 defined as:
   All systems are either BILAG C or BILAG D/E provided that at baseline at least one system was BILAG A or 2 systems were BILAG B
Proportion of patients achieving a response by SLEDAI-2K Responder Index 50 (SRI-50) at week 26 defined as a minimum clinically important difference (MCID) of one (Touma et. al., 2012)
Proportion of subjects who achieve a response at week 26 by composite SLE Responder Index (cSRI) defined as BILAG 2004 SR and no deterioration by SLEDAI-2K
Proportion of subjects achieving a response by the SLE Responder Index at week 26, defined as ≥4 point improvement in the SELENA-SLEDAI along with no new BILAG A or no more than 1 new BILAG B score, and no worsening (0.3 point increase) in the physician global assessment (PGA).
Mean change from baseline in SF-36 Physical Component Summary (PCS) and Mental Component Summary (MCS),
   if one or both are statistically significant then each of the 8 domains may be examined for statistical significance without a p value correction.

### Exploratory Efficacy Variables

Time to first confirmed severe SLE flare defined as a new BILAG “A” score in any organ system or 2 new “B”s (Gordon et al., 2003)
Time to first confirmed major SLE flare defined by the “Fortin” definition of major flare:
   Initiation or increase of immunosuppressive or high-dose glucocorticoid therapy
   Hospitalization or
   Death due to SLE
Change from baseline at week 26 in the cumulative damage index as measured by Systemic Lupus International Collaborating Clinics/American College of Rheumatology Damage Index (SLICC/ACR DI)
Change from baseline at week 26 in daily glucocorticoid dose

### Patient Reported Outcomes (PROs):

Change from baseline in subject global assessment of disease activity
Proportion of subjects within each treatment group reporting improvements ≥MCID=10 on a VAS scale of 100. (MCID=minimum clinically important differences for improvement and/or no deterioration)
Mean change from baseline in SF-36 Physical Component Summary (PCS) and Mental Component Summary (MCS), if one or both are statistically significant then each of the 8 domains may be examined for statistical significance without a p value correction
Proportion of patients within each treatment group reporting “no deterioration” based on not exceeding MCID values for worsening [summary scores −0.8; domain scores −2.5];
Proportion of patients within each treatment group reporting improvements ≥MCID [summary scores 2.5; domain scores 5.0;
Mean changes from baseline in Lupus-QOL within each treatment group

### Safety Variables

The safety and tolerability of therapy with Edratide will be determined by reported AEs, physical examinations, vital signs, and laboratory tests. Subjects who receive any study medication (even a partial dose) are considered evaluable for safety.

### Clinical Laboratory

The central laboratory is responsible to ship, store, analyze and report all laboratory safety tests performed for this study. Laboratory data from the central laboratory will be reported to the investigator and sponsor by fax within 24 hours, followed by NCR hard copies sent to the investigator.

In cases where the results may be of urgent clinical significance, the laboratory will contact the investigator by phone and fax the results to the sponsor.

The subject will attend the clinical unit preferably during the morning at Baseline and at the scheduled visits.

Safety laboratory tests will be performed at:
Screening visit
Baseline visit
Monthly visits 1, 2, 3, 4, 5 and 6.
At any unscheduled visit if deemed necessary

List of laboratory tests:
Hematology:
   CBC
   Prothrombin Time (INR and %)
   Partial Thromboplastin Time (PTT)
Clinical Chemistry:
   Alkaline phosphatase
   Alanine aminotransferase (ALT/SGPT)
   Aspartate aminotransferase (AST/SGOT)
   γ-glutamyl transpeptidase (GGT
   Total bilirubin
   Lactate Dehydrogenase (LDH)
   CPK
   Creatinine
   Blood Urea Nitrogen
   Glucose
Electrolytes:
   Sodium
   Potassium
   Chloride
   Calcium
   phosphorus
Proteins:
   Total protein
   Albumin
   globulin
Uric acid
CRP
ESR
anti-dsDNA, C3 and C4

Urinalysis
Glucose
Ketones
Blood, Leukocytes
Specific gravity
pH
Nitrite
Bilirubin
Urobilinogen
Urine Protein
Urine Creatinine
Microscopic examination of urinary sediments (including RBC/HPF, WBC/HPF, RBC and WBC casts)
Urine protein creatinine ratio (PCR)

Urine will be examined by a dipstick test (performed at the site) for:
Glucose
Ketones
Blood, leukocytes
Protein
Specific gravity
pH
Nitrite
Bilirubin
Urobilinogen
β-hCG test for pregnancy: Will be performed for female subjects of childbearing potential at every study visit.

Serum Virology at screening visit only:
Hepatitis B surface antigen (HBsAg), HBsAb, HBcore Ab
HCV Antibody

All tests results should be within acceptable ranges for the inclusion of SLE patients in the study.

At all study visits, should the dipstick test have been positive for protein, leukocytes, nitrite and/or erythrocytes, an additional mid-stream sample of urine will be sent for bacteriological culture.

Urinalysis will be performed at all scheduled visits but in case of menses, the urine will not be collected as scheduled but a few days after/before visit. Visits should be scheduled to a menses free period (in accordance with the visits schedule and windows generated by the IVRS).

### Immunology Tests

To evaluate the immunological responses following multiple, once weekly, subcutaneous injections of Edratide, blood sampling for several immunology tests will be taken at weeks 8, 12 and 26 and stored at the central laboratory for future testing. Future testing will only be performed if the study meets the primary endpoint.
Disease-related immunological tests:
   Anti-dsDNA antibodies, C3, C4 and CH50 will be performed at baseline and at every visit until week 26.
   Anti-ENA antibodies (anti-Ro, anti-La, anti-RNP, anti-Sm) and direct Coombs' test will be performed at baseline (pre-dose) and week 26.
   Total IgG, IgM and IgA, Anti-cardiolipin antibodies IgG and IgM, anti-beta2 GPI antibodies IgG and IgM and lupus anticoagulant tests will be performed at baseline (pre-dose) and week 26.
Exploratory immunological tests:
Blood samples will be collected and stored at the central lab. They may be analysed if the study meets its primary endpoint. Analysis may include:
   Anti-Edratide antibodies may be performed at baseline and at weeks 12 and 26

### Pharmacokinetic Study (PK)

Plasma levels of Edratide and SBECD will be evaluated in a subset of sites and assessed at baseline, after the first administration of study drug and at Week 26.

PK sample for Edratide

Five (5) ml blood will be collected at each time point before and after the first administration (baseline) and at visit Weeks 12 and 26.

Blood samples will be collected at the following time points after the first administration and at monthly visits 3 and 6: pre-dose and at 5, 10, 20, 40, 60 minutes post dose.

Subjects participating in this sub study will be dosed with their weekly study drug at study site at these visits.

PK sample for CAPTISOL®

The aim of this ancillary study is to assess the clearance of CAPTISOL® and the extent of its accumulation after 3 and 6 months of treatment.

Three (3) ml blood will be collected at each time point before and after the first administration (baseline) and at monthly visits 3 and 6.

Blood samples will be collected at the following time points after the first administration and at monthly visits 3 and 6: pre-dose and at 5, 10, 20, 40 minutes and 1, 2, 4, 8 and 24 hours post dose.

Subjects participating in this sub study will be dosed with their weekly study drug at study site at these visits.

The actual time point will be recorded in the CRF.

28 ml of blood will be taken at baseline, and the rest throughout the study.

### Adverse Events

### Adverse Events (AE) and Serious Adverse Events (SAE)

An adverse event (AE) is any untoward medical occurrence in a subject participating in a clinical trial. An adverse event can be any unfavorable and unintended sign, symptom or disease temporally associated with the use of the study medication, whether or not considered related to the study medication. AEs will be collected from the start of treatment until 30 days following the final visit dose. Any events occurring prior to treatment will be recorded on the medical history page with the event name and onset date and end date if not continuing. Pre-existing, known clinically significant conditions observed at screening should be recorded as medical history.

This definition also includes accidental injuries, reasons for any change in medication (drug and/or dose) other than planned titration, reasons for admission to a hospital, or reasons for surgical procedures (unless for minor elective surgery for a pre-existing condition). It also includes adverse events commonly observed and adverse events anticipated based on the pharmacological effect of the study medication. Any laboratory abnormality assessed as clinically significant by the Investigator must be recorded as an adverse event.

A treatment emergent adverse event is any adverse event occurring after start of study medication and within the time of residual drug effect, or a pre-treatment adverse event or pre-existing medical condition that worsens in intensity after start of study medication and within the time of residual drug effect.

Adverse events should be recorded as diagnoses, if available. If not, separate sign(s) and symptom(s) are recorded. One diagnosis/symptom should be entered per record.

Note that death is not an event, but the cause of death is. An exception is the event of sudden death of unknown cause. Note that hospitalization is not an event; however, the reason for hospitalization is. Procedures are not events; the reasons for conducting the procedures are. In general, only the reason for conducting the procedure will be captured as an adverse event. However, if deemed necessary by the Investigator, a procedure can be captured along with the reason for conducting the procedure.

An overdose or medication error is not an adverse event unless it is temporally associated with an unfavourable or unintended sign or symptom.

Each AE is to be classified by the investigator as serious or non-serious. A serious adverse event (SAE) is any untoward medical occurrence or effect that occurs at any dose:
Results in death
Is life-threatening (i.e., an immediate risk of death)
Requires in-patient hospitalization or prolongation of existing hospitalization
Results in persistent or significant disability/incapacity
Is associated with a congenital anomaly/birth defect
Is an important medical event

An adverse event caused by an overdose or medication error is considered serious if a criterion listed in the definition above is fulfilled.

Important adverse events that may not result in death, may not be life-threatening, or do not require hospitalization may be considered serious when, based upon appropriate medical judgment, they may jeopardize the subject's safety or may require medical or surgical intervention to prevent one of the outcomes listed above.

Serious adverse events also include any other event that the investigator or sponsor judges to be serious or which is defined as serious by the regulatory agency.

The investigator is to report all directly observed adverse events and all adverse events spontaneously reported by the trial subject using concise medical terminology. In addition, each trial subject will be questioned about adverse events. The question asked will be “Since you began taking the study medication, have you had any health problems?”

Procedures for Assessing, Recording, and Reporting Adverse Events and Serious Adverse Events

Throughout the duration of the study, the Investigator will closely monitor each subject for evidence of drug intolerance and for the development of clinical or laboratory evidence of adverse events. All adverse events (expected or unexpected) which occur during the course of the study, whether observed by the Investigator or by the subject, and whether or not thought to be drug-related, will be reported and followed until resolution or until they become stable.

The description of the adverse event will include description of event, start date, stop date, intensity, if it was serious, relationship to test drug, change in test drug dosage, if the subject died, and if treatment was required.

Events will be coded to one of the following intensity categories below:

| Severity | Definition |
|---|---|
| Mild | Awareness of signs or symptoms, but no disruption |
| | of usual activity |
| Moderate | Event sufficient to affect usual activity |
| | (disturbing) |
| Severe | Event causes inability to work or perform usual |
| | activities (unacceptable) |

Events will be coded into one of the following causality categories as defined below:

| Relationship | Category | Description |
|---|---|---|
| Unrelated to | Unrelated | Clearly and incontrovertibly due only |
| investigational to | | extraneous causes, and does not |
| agent | | meet criteria listed under possible |
| | | or probable. |
| | Unlikely | Does not follow a reasonable temporal |
| | | sequence from administration. May |
| | | have been produced by the subject's |
| | | clinical state or by environmental |
| | | factors or other therapies |
| | | administered. |
| Related to | Possible | Follows a reasonable temporal |
| investigational | | sequence from administration, but may |
| agent | | have been also produced by the |
| | | subject's clinical state, |
| | | environmental factors or other |
| | | therapies administered. |
| | Probable | Clear-cut temporal association with |
| | | administration with improvement on |
| | | cessation of investigational |
| | | medicinal product or reduction in |
| | | dose. Reappears upon rechallenge. |
| | | Follows a known pattern of response |
| | | to the investigational medicinal |
| | | product. |

Adverse events with the causality assessed as unrelated or unlikely are categorized as not related to study medication.

Adverse events with the causality assessed as possible or probable are categorized as related to study medication and are called adverse drug reactions.

### Description of Study Procedures

Informed consent: all subjects must sign and date informed consent personally. Consent by a guardian is not acceptable unless the subject is unable to sign due to a physical problem with a hand or arm. In that case, the subject must give verbal consent, which must be witnessed by an observer not directly involved in the study. Subjects not mentally competent to consent on their own are not permitted to participate. The consent process needs to be properly documented in the source data.

Medical history and physical examination: full medical history, including details of SLE diagnosis, treatment and course, and physical during screening; brief physical examination of pertinent systems and interim history at other visits.

All physical examinations are to include vital signs and weight. Height will be collected only at the baseline physical examination.

Baseline tests: 12-lead ECG, PA and lateral CXR. A historical CXR done up to 1 month prior to study entry is acceptable.

β-hCG: for women of childbearing potential, pregnancy test throughout the study.

Complete blood count (CBC): including routine red cell parameters, total WBC count, absolute values for individual white blood cell types, platelet count, and reticulocyte count.

Coagulation profile: INR, aPTT.

Urinalysis: see above

Immunology tests: see above

Concomitant medications: At baseline, a complete history of all treatment for SLE, as well as all medications, both prescription and OTC, taken within 30 days prior to the screening visit. On study, all medications, both prescription and OTC, are to be recorded. Indications for each medication should be collected, including whether the medication was taken prophylactically or therapeutically. With the exception of analgesics and other medications taken for treatment of adverse reactions, actual doses and frequencies of administration need not be recorded, other than notation as to whether a medication was taken regularly or as required (PRN). PRN medications which were prescribed but not actually taken should not be recorded.

Complete dosing information (dosage and actual number of doses taken each day), for all medications taken for treatment of adverse reactions to study treatment is to be recorded.

Special attention to the recording of steroid dosing should be given. Subject's steroid dosing diaries must be transcribed to the eCRF in full detail.

Adverse events: patients will be questioned regarding the occurrence or resolution of adverse events.

Disease assessment:
BILAG 2004
SLEDAI-2K
SLEDAI-2K Responder Index 50 (SRI-50)
Patient reported short-form quality of life assessment (SF-36)
Lupus-specific quality of life form Lupus-QOL, PGA
Damage index: SLICC/ACR disease index
Cumulative glucocorticoid dose

Tolerability assessment:
Time to withdrawal from treatment
Number of subjects who discontinued study
Number of subjects who discontinued study due to AEs/SAEs
Treatment compliance

Safety assessments:
Vital signs, physical examinations, routine laboratory safety tests, adverse events

PK assessments:

Plasma levels of Edratide and CAPTISOL® will be assessed in a subset of sites at baseline, weeks 12 and 26.

### Results

Treatment with one 0.25 mg dose of Edratide every week to subjects with SLE results in improvements of disease parameter/s in specific sub-populations of subjects included in the trial.

Some of the sub-populations in which disease improvement is achieved are: subjects who have at least one organ system categorized as BILAG A or at least two organ systems categorized as BILAG B; subjects who have SLEDAI-2K score of 6 or higher; subjects receiving 15 mg/day or less prednisone or prednisone equivalent; and/or subjects whose prednisone or prednisone equivalent dose is gradually tapered from 15 mg/day or less to 7.5 mg/day or less.

### Primary Outcome Measure:

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant proportion of the subjects having at least one organ system categorized as BILAG A or B at baseline dropping by one score, without deterioration to BILAG A or B in any other system at week 26.

### Secondary Outcome Measures:

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant proportion of the subjects having all organ systems either BILAG C or BILAG D/E when measured at week 26.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant proportion of the subjects achieving a minimum clinically important difference (MCID) of one measured by SLEDAI-2K Responder Index 50 at week 26.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant proportion of the subjects achieving a response at week 26 measured by composite SLE Responder Index (cSRI), which is defined as having all organ systems either BILAG C or BILAG D/E when measured by BILAG 2004 and no deterioration measured by SLEDAI-2K at week 26.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant proportion of the subjects achieving response at week 26 measured by the SLE Responder Index, defined as equal or greater than 4 point improvement in the SELENA-SLEDAI, wherein the subjects have no new organ system categorized as BILAG A or no more than one organ system categorized as BILAG B, and wherein the subjects have less than 0.3 point increase in the physician global assessment.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant change of SF-36 Physical Component Summary (PCS) and Mental Component Summary (MCS) relative to baseline.

### Exploratory Outcome Measures:

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significantly increased mean time to first confirmed severe SLE flare defined as any organ system newly categorized as BILAG A or two organ systems newly categorized as BILAG B.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significantly increased mean time to first confirmed major SLE flare defined as initiation or increase of immunosuppressive or high-dose glucocorticoid therapy, hospitalization or death due to SLE.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant change in mean cumulative damage index as measured by SLICC/ACR DI at week 26 relative to baseline.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant change in mean glucocorticoid dose at week 26 relative to baseline.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant improvement in mean Lupus-QOL relative to baseline.

Treatment with one 0.25 mg dose of Edratide every week to subjects with SLE (i) who have at least one organ system categorized as BILAG A or at least two organ systems categorized as BILAG B; (ii) who have SLEDAI-2K score of 6 or higher; (iii) receiving 15 mg/day or less prednisone or prednisone equivalent at baseline; (iv) whose prednisone or prednisone equivalent dose is gradually tapered from 15 mg/day or less to 7.5 mg/day or less; or any combination thereof, results in significant improvement of global assessment of disease activity based on minimum clinically important differences (MCID).

### Discussion

Previous clinical trials with the product Edratide did not employ a weekly dose smaller than 0.5 mg, which was determined as the optimal dose in the Prelude study. Based on earlier studies, a weekly dose smaller than 0.5 mg per subject was not expected to be effective. Furthermore, preclinical studies in animal models that tested various doses of Edratide indicated that a human dose beneath 0.5 mg was not expected to be effective.

It was unexpectedly found and disclosed herein for the first time, that a weekly injection dose of 0.25 mg of the peptide Edratide (hCDR1) to a patient suffering from SLE, results in improvement of at least one disease parameter. In particular, it is herein disclosed that a weekly 0.25 mg dose of Edratide is effective in treating a sub-population of SLE subjects selected from: subjects who have at least one organ system categorized by the British Isles Lupus Assessment Group 2004 (BILAG 2004) as BILAG A or at least two organ systems categorized as BILAG B; subjects who have SLE disease activity index (SLEDAI-2K) score of 6 or higher; and/or subjects receiving 15 mg/day or less prednisone or prednisone equivalent. In addition, it is herein disclosed that a weekly 0.25 mg dose of Edratide is effective in enabling reduction of the amount of corticosteroid therapy required by a human subject to control disease activity of SLE.

### REFERENCES

1. American College of Rheumatology (ACR). (1999). Guidelines for referral and management of systemic lupus erythematosus in adults. Arthritis Rheum. 42(9):1785-96.
2. Bailey, T., Rowley K., Bernkopf, A. (2011). A review of systemic lupus erythematosus and current treatment options. Form. J. 46:178-194.
3. Danchenko, N., Satia, J. A., Anthony, M. S. (2006). Epidemiology of systemic lupus erythematosus: A comparison of worldwide disease burden. Lupus. 15 (5): 308-318.
4. Dayal et al., (2002). SLE/myositis overlap: are the manifestations of SLE different in overlap disease?. Lupus. 11(5):293-8.
5. Fairhurst et al., (2006). Systemic lupus erythematosus: multiple immunological phenotypes in a complex genetic disease. Adv. Immunol., 92:1-69.
6. Gladman et al., (2002). Systemic lupus erythematosus disease activity index 2000. Feb; 29(2):288-91.
7. Gordon, C., Sutcliffe, N., Skan, J., Stoll, T. Isenberg, D. A. (2003). Definition and treatment of lupus flares measured by the BILAG index. Rheumatology. 42:1372-1379
8. Gottschalk et al., (2015). Pathogenic Inflammation and Its Therapeutic Targeting in Systemic Lupus Erythematosus. Front Immunol., Oct 28; 6:550.
9. Harley et al., (2008). Genome-wide association scan in women with systemic lupus erythematosus identifies susceptibility variants in ITGAM, PXK, KIAA1542 and other loci. Nat Genet., 40, 204-210
10. Haubitz M., (2010). New and emerging treatment approaches to lupus. Biologics, Sep 13; 4:263-71.
11. Ioannou et al., (2002). Current concepts for the management of systemic lupus erythematosus in adults: a therapeutic challenge. Postgrad Med J. Oct;78(924):599-606.
12. Isenberg, D. et al., (2005). Development and initial validation of an updated version of the British Isles Lupus Assessment Group's disease activity index for patients with systemic lupus erythematosus. Rheumatology. 44:p02-906.
13. Manson, J. J. and Rahman, A., (2006). Systemic Lupus Erythematosus. Orphanet J Rare Dise. 1:6.
14. Petri M., (1998). Treatment of systemic lupus erythematosus: an update. Am Fam Physician, Jun; 57(11):2753-60.
15. Petri, M., et al., (2012). Derivation and validation of Systemic Lupus International Collaborating Clinics classification criteria for Systemic Lupus Erythematosus (SLICC). Arthritis Rheum. 64(8):2677-2686.
16. Ruiz-Irastorza et al., (2012). Glucocorticoid use and abuse in SLE. Rheumatology (Oxford) Jul; 51(7):1145-53.
17. Sthoeger et al., (2014). Novel approaches to the development of targeted therapeutic agents for systemic lupus erythematosus. Journal of Autoimmunity, Nov; 54:60-71.
18. Touma, Z. et al., (2012). Systemic Lupus Erythematosus Disease Activity Index 2000 Responder Index 50: sensitivity to response at 6 and 12 months. Rheumatology. 51(10):1814-1819.
19. Urowitz, M. B., Isenberg, D. A., Wallace, D. J. (2015). Safery and efficacy of hCDR1 (Edratide) in patients wich active systemic lupus erythematosus: results of phase II study. Lupus Science & Medicine. 2:e000104.
20. Waisman, A. et al., (1997). Modulation of murine systemic lupus erythematosus with peptides based on complementarity determining regions of a pathogenic anti-DNA monoclonal antibody. Proc. Natl. Acad. Sci. U.S.A., Apr 29; 94(9):4620-5.
21. Ware J E et al., (1992). The MOS 36-item short-form health survey (SF-36). I. Conceptual framework and item selection. Medical Care 30:473-483.
22. Yee, C. S. et al., (2010). Numerical scoring for the BILAG-2004 index. Rheumatology. 49:1665-1669.

What is claimed is:

1. A method of reducing the amount of corticosteroid dose received by a human subject who is suffering from systemic lupus erythematosus (SLE) to an amount less than the amount received by the human subject for effective control of SLE disease activity when only the corticosteroid drug is administered to the human subject, which comprises periodic administration to the human subject of one subcutaneous injection of 0.25 mg dose of Edratide every week and said reduced amount of corticosteroid drug in a manner effective to treat SLE.

2. The method of claim 1, further comprising gradually reducing the amount of corticosteroid drug after baseline.

3. The method of claim 1, wherein the corticosteroid drug is prednisone or prednisone equivalent.

4. The method of claim 3, wherein the amount of prednisone or prednisone equivalent at baseline is 15 mg/day or less.

5. The method of claim 3, further comprises gradually reducing the amount of prednisone or prednisone equivalent to 7.5 mg/day or less.

6. The method of claim 1, wherein the human subject has been administered an amount of prednisone or prednisone equivalent for at least 14 days before baseline.

7. The method of claim 1, wherein the subject suffers from systemic lupus erythematosus (SLE) and having (a) at least one organ system categorized by the British Isles Lupus Assessment Group 2004 (“BILAG”) as category A (“BILAG A”); or (b) at least two organ systems categorized as BILAG B; or (c) SLEDAI-2K score of 6 or higher.

8. The method of claim 7, wherein the lupus disease activity is measured by a disease activity score selected from the group consisting of British Isles Lupus Assessment Group 2004 (BILAG), SLE disease activity index (SLEDAI-2K), SLEDAI-2K Responder Index 50 (SRI-50), composite SLE Responder Index (cSRI), minimum clinically important differences (MCID), patient reported short-form quality of life assessment (SF-36) Physical Component Summary (PCS) and/or Mental Component Summary (MCS), and lupus-specific quality of life form (Lupus-QOL) or a combination thereof.

9. The method of claim 7, wherein the method results in the subject who had at least one organ system categorized as BILAG A or at least two organ systems categorized as BILAG B at baseline:
having reduction in the one organ system categorized as BILAG A or in the at least two organ systems categorized as BILAG B by one score in any one organ system, without having any other organ systems deteriorated to BILAG A or B; or
having all organ systems categorized as either BILAG C or BILAG D/E after treatment; or
having all organ systems categorized as either BILAG C or BILAG D/E after treatment, and no deterioration measured by SLEDAI-2K after treatment.

10. The method of claim 7, wherein the method results in the subject having equal or greater than 4 point improvement in the SELENA-SLEDAI, wherein the subject having no new organ system categorized as BILAG A or no more than one organ system categorized as BILAG B, and wherein the subject having less than 0.3 point increase in the physician global assessment.

11. The method of claim 7, wherein the method results in the subject having increased time to first confirmed severe SLE flare or time to first confirmed major SLE flare.

12. The method of claim 7, wherein the method results in the subject having at least one of:
a minimum clinically important difference (MCID) of one for SRI-50;
a significant change in SF-36 PCS and/or MCS relative to baseline;
a significant change in daily glucocorticoid dose;
a significant improvement in Lupus-QOL; and
a significant improvement of global assessment of disease activity based on minimum clinically important differences (MCID).

13. The method of claim 1, wherein the subject has a documented medical history to meet Systemic Lupus International Clinics (SLICC) classification criteria for SLE, or has accumulated 4 classification criteria of the American College of Rheumatology for SLE.

14. The method of claim 1, wherein the subject has been on stable SLE treatment regimen for at least three months at baseline.

15. The method of claim 1, wherein the subject receives 2 g/day or less of mycophenolate mofetil.

16. The method of claim 1, wherein 0.25 mg dose of Edratide contains 0.25 mg of Edratide free base.

17. The method of claim 1, wherein 0.25 mg dose of Edratide contains about 0.275 mg of Edratide acetate.

18. The method of claim 1, wherein the subcutaneous injection of 0.25 mg dose of Edratide further comprises 120 mg of hepta-(sulfobutyl ether)-β-cyclodextrin.

19. The method of claim 1, wherein said reducing the amount of corticosteroid results in reduction of an adverse effect of chronic steroid treatment selected from the group consisting of cushingoid habitus, central obesity, hypertension, infection, capillary fragility, hirsutism, accelerated osteoporosis, cataracts, diabetes mellitus, myopathy and psychosis.

20. A kit or package which comprises:
a) one or more unit doses of 0.25 mg dose of Edratide; and
b) instructions for use of a) for periodically dispensing 0.25 mg dose of Edratide to a human subject who is suffering from systemic lupus erythematosus (SLE), and
wherein the human subject has at least one organ system categorized by the British Isles Lupus Assessment Group 2004 (“BILAG”) as category A (“BILAG A”) or at least two organ systems categorized as BILAG B; wherein the human subject has SLEDAI-2K score of 6 or higher; wherein the human subject is administered an amount of corticosteroid drug; and/or wherein the amount of corticosteroid dose received by a human subject is reduced to an amount less than the amount received by the human subject for effective control of SLE disease activity when only the corticosteroid drug is administered to the human subject.
